SEQUENCE LISTING (40 sequences)

SEQ ID NO: 1 - protein, 450 aa, Homo sapiens
MEGSKTSNNSTMQVSFVCQRCSQPLKLDTSFKILDRVTIQELTAPLLTTAQAKPGETQEEETNSGEEPFIETPRQDGVSRRFIPPARMMSTESANSFTLIGEASDGGTMENLSRRLKVTGDLFDIMSGQTDVDHPLCEECTDTLLDQLDTQLNVTENECQNYKRCLEILEQMNEDDSEQLQMELKELALEEERLIQELEDVEKNRKIVAENLEKVQAEAERLDQEEAQYQREYSEFKRQQLELDDELKSVENQMRYAQTQLDKLKKTNVFNATFHIWHSGQFGTINNFRLGRLPSVPVEWNEINAAWGQTVLLLHALANKMGLKFQRYRLVPYGNHSYLESLTDKSKELPLYCSGGLRFFWDNKFDHAMVAFLDCVQQFKEEVEKGETRFCLPYRMDVEKGKIEDTGGSGGSYSIKTQFNSEEQWTKALKFMLTNLKWGLAWVSSQFYNK

SEQ ID NO: 2 - protein, 9 aa, Artificial
  Synthetic Formula I
  misc_feature (1)..(1): Xaa can be any naturally occurring amino acid
  misc_feature (6)..(9): Xaa can be any naturally occurring amino acid
XNATFXXXX

SEQ ID NO: 3 - protein, 9 aa, Artificial
  Synthetic Peptide 3
FNATFHIWH

SEQ ID NO: 4 - protein, 11 aa, Artificial
  Synthetic Peptide 4
VFNATFEIWHD

SEQ ID NO: 5 - protein, 11 aa, Artificial
  Synthetic Peptide 5
CFNATFEIWHD

SEQ ID NO: 6 - protein, 11 aa, Artificial
  Synthetic Peptide 6
VWNATFEIWHD

SEQ ID NO: 7 - protein, 11 aa, Artificial
  Synthetic Peptide 7
VFNATFDIWHD

SEQ ID NO: 8 - protein, 11 aa, Artificial
  Synthetic Peptide 8
VFNATFELWHD

SEQ ID NO: 9 - protein, 11 aa, Artificial
  Synthetic Peptide 9
VFNATFEIFHD

SEQ ID NO: 10 - protein, 11 aa, Artificial
  Synthetic Peptide 10
VFNATFEIWYD

SEQ ID NO: 11 - protein, 11 aa, Artificial
  Synthetic Peptide 11
VFNATFEIWHE

SEQ ID NO: 12 - protein, 11 aa, Artificial
  Synthetic Peptide 12
VWNATFELWHD

SEQ ID NO: 13 - protein, 11 aa, Artificial
  Synthetic Peptide 13
VFNATFEVWHD

SEQ ID NO: 14 - protein, 11 aa, Artificial
  Synthetic Peptide 14
VLNATFEIWHD

SEQ ID NO: 15 - protein, 11 aa, Artificial
  Synthetic Peptide 15
VFNATFEMWHD

SEQ ID NO: 16 - protein, 11 aa, Artificial
  Synthetic Peptide 16
VWNATFHIWHD

SEQ ID NO: 17 - protein, 11 aa, Artificial
  Synthetic Peptide 17
VFNATFEFWHD

SEQ ID NO: 18 - protein, 11 aa, Artificial
  Synthetic Peptide 18
VFNATFEYWHD

SEQ ID NO: 19 - protein, 11 aa, Artificial
  Synthetic Peptide 19
VFNATFERWHD

SEQ ID NO: 20 - protein, 10 aa, Artificial
  Synthetic Peptide 20
FNATFEIWHD

SEQ ID NO: 21 - protein, 10 aa, Artificial
  Synthetic Peptide 21
VFNATFEIWH

SEQ ID NO: 22 - protein, 9 aa, Artificial
  Synthetic Peptide 22
FNATFEIWH

SEQ ID NO: 23 - protein, 9 aa, Artificial
  Synthetic Peptide 23
WNATFHIWH

SEQ ID NO: 24 - protein, 10 aa, Artificial
  Synthetic Peptide 24
VWNATFHIWH

SEQ ID NO: 25 - protein, 10 aa, Artificial
  Synthetic Peptide 25
WNATFHIWHD

SEQ ID NO: 26 - protein, 15 aa, Artificial
  Synthetic Formula II
  MISC_FEATURE (7)..(7): E or H
  MISC_FEATURE (11)..(11): D or S
  MISC_FEATURE (13)..(13): Q or E
VFNATFXIWHXGXFG

SEQ ID NO: 27 - protein, 15 aa, Artificial
  Synthetic Peptide 27
VFNATFEIWHDGEFG

SEQ ID NO: 28 - protein, 15 aa, Artificial
  Synthetic Peptide 28
VFNATFHIWHSGQFG

SEQ ID NO: 29 - protein, 18 aa, Artificial
  Synthetic Formula III
  MISC_FEATURE (9)..(9): E or H
  MISC_FEATURE (13)..(13): D or S
  MISC_FEATURE (15)..(15): W or E
  MISC_FEATURE (15)..(15): Q or E
TNVFNATFXIWHXGXFGT

SEQ ID NO: 30 - protein, 18 aa, Artificial
  Synthetic Peptide 30
TNVFNATFEIWHDGEFGT

SEQ ID NO: 31 - protein, 18 aa, Artificial
  Synthetic Peptide 31
TNVFNATFHIWHSGQFGT

SEQ ID NO: 32 - protein, 10 aa, Artificial
  Synthetic RGD-4C
CCDCRGDCFC

SEQ ID NO: 33 - protein, 6 aa, Artificial
  Synthetic NGR
CCNGRC

SEQ ID NO: 34 - protein, 9 aa, Artificial
  Synthetic LyP-1
CGNKRTRGC

SEQ ID NO: 35 - protein, 7 aa, Artificial
  Synthetic SMS
SMSIARL

SEQ ID NO: 36 - protein, 11 aa, Artificial
  Synthetic TAT
YGRKKRRQRRR

SEQ ID NO: 37 - protein, 31 aa, Artificial
  Synthetic TAT-Beclin 1
YGRKKRRQRRRGGTNVFNATFEIWHDGEFGT

SEQ ID NO: 38 - protein, 31 aa, Artificial
  Synthetic Scramble TAT-Beclin 1
YGRKKRRQRRRGGVGNDFFINHETTGFATEW

SEQ ID NO: 39 - DNA, 59 bp, Artificial
  Synthetic Sense
tgcgggagtatagtgagtttaattcaagagattaaactcactatactcccgcttttttc

SEQ ID NO: 40 - DNA, 63 bp, Artificial
  Synthetic Anti-sense
tcgagaaaaaagcgggagtatagtgagtttaatctcttgaattaaactcactatactcccgca

(19) United States
(12) Patent Application Publication
(10) Pub. No.: US 2021/0379146 A1
(43) Pub. Date: Dec. 9, 2021

(54) METHOD TO RESTORE OR IMPROVE COGNITIVE FUNCTIONS

(71) Applicants: INSERM (INSTITUT NATIONAL DE LA SANTÉ ET DE LA RECHERCHE MÉDIGALE), Paris (FR); UNIVERSITÉ DE PARIS, Paris (FR); CENTRE NATONAL DE LA RECHERCHE SCIENTFIQUE (CNRS), Paris (FR)

(72) Inventors: Franck OURY, Paris (FR); Patrice CODOGNO, Paris (FR)

(21) Appl. No.: 17/282,474

(22) PCT Filed: Oct. 3, 2019
(86) PCT No.: PCT/EP2019/076796

§ 371 Date: Apr. 2, 2021

(30) Foreign Application Priority Data
Oct. 4, 2018 (EP) 18306307.2

(51) Int. Cl. A61K 38/17; A61P 25/26

(52) U.S. Cl. CPC A61K 38/1709; A61P 25/26

(57) ABSTRACT

The present invention relates to the field of memory and cognitive functions. Here the inventors show that memory stimulations induce autophagy in the mouse hippocampus, while local pharmacological and genetic modulations of hippocampal autophagy strongly influence memory acquisition. More, the inventors observe that hippocampal autophagy declines during aging and they find that restoring autophagy specifically in the hippocampus of aged mice, following autophagy inducers (such as TAT-Beclin-1), can significantly reverse age-related memory decline. Their results reveal a novel physiological role of autophagy in regulating hippocampal-dependent memory functions, and demonstrate the potential therapeutic benefits of modulating autophagy in order to prevent and/or reverse the deleterious effects of aging on cognitive function. The present invention relates to an activator of the autophagy for use in the restoration and/or improvement of cognitive functions in a subject in need thereof.

### FIELD OF THE INVENTION

The present invention relates to an activator of the autophagy for use in the restoration and/or improvement of cognitive functions in a subject in need thereof.

### BACKGROUND OF THE INVENTION

Normal brain aging is characterized by a progressive decline in cognitive functions that start to develop around midlife. One of the most commonly affected regions of the brain is the hippocampus, which leads to learning deficits and memory impairments. According to a report by the World Health Organization, the number of people aged 65 or older is expected to grow by nearly 1.5 billion in 2040, representing 16 percent of world's population. Indeed, a significant proportion of individuals will have to cope with alterations in memory function that are associated with normal aging. Therefore, a deeper understanding of how a healthy brain ages would profit from the identification of mechanisms involved in age-related cognition deficits and may lead to the development of novel therapeutic strategies that prevent the decline and/or restore the resilience of our mental health-span during aging.

The hippocampus of the mammalian brain is pivotal for the control of learning and memory. The integration and consolidation of memory throughout life relies on the capacity of the hippocampus for neuronal structural reorganization and plasticity (5, 6). This is mainly characterized by continuous rearrangement in dendritic and synaptic complexity, neurotransmission, and by the generation of newborn neurons through the adult neurogenesis (5, 7, 8). However, this high degree of hippocampal neuronal and synaptic rearrangement is also one of the major reasons for its extreme vulnerability during aging. Indeed, a hallmark of hippocampal aging is that age-related cognitive decline correlates with a reduction in neuronal plasticity (5, 9, 10). This decline leads to a progressive impairment of hippocampal-dependent learning and memory (5, 6, 10), which is characterized by deficits in episodic memory, attention, working memory, and spatial learning (10, 11). With the progressive increase in life expectancy in developed societies, the number of individuals affected by age-related memory loss is bound to increase. Therefore, a deeper understanding of the cellular mechanism controlling hippocampal-dependent memory acquisition and age-related cognition deficits are now fundamental.

To address this question the inventors focused on macroautophagy (hereafter called autophagy), a cellular catabolic process whereby proteins and organelles are engulfed in double-membrane vesicles called autophagosomes (AP) and then transported to lysosome for degradation (1, 3, 12). The biogenesis of AP is orchestrated by multiple signaling pathways and dynamic membrane complexes, which contain autophagy-related proteins (ATG) and associated proteins such as VPS34, VPS15 and AMBRA1 (1, 2). The ATG5-ATG12-ATG16L1 complex allows for the lipidation of the LC3 protein (LC3-II) which is then recruited to the site(s) of nascent AP to favor formation and maturation of double membrane structure (13). Importantly, autophagy plays also a fundamental role in stress-response mechanisms (1, 12) to various physiological stimuli (1, 2) and for the maintenance of regenerative capacity of stem cell progenitors (14, 15).

### SUMMARY OF THE INVENTION

Here the inventors show that memory stimulations induce autophagy in the mouse hippocampus, while local pharmacological and genetic modulations of hippocampal autophagy strongly influence memory acquisition. These effects are associated with an induction of neuronal synaptic plasticity and dendritic spine formation in response to memory stimuli. Importantly, they observe that hippocampal autophagy declines during aging and they find that restoring autophagy specifically in the hippocampus of aged mice, following autophagy inducers (such as TAT-Beclin-1), can significantly reverse age-related memory decline. Lastly, they demonstrate that autophagy mediates the recently described beneficial effect of young plasma on memory function during aging.

Their results reveal a novel physiological role of autophagy in regulating hippocampal-dependent memory functions, and demonstrate the potential therapeutic benefits of modulating autophagy in order to prevent and/or reverse the deleterious effects of aging on cognitive function, which is now a highly-prioritized challenge of healthcare systems in western societies.

Thus, the present invention relate to an activator of the autophagy for use in the restoration and/or improvement of cognitive functions in a subject in need thereof.

### DETAILED DESCRIPTION OF THE INVENTION

A first aspect of the invention relates to an activator of the autophagy for use in the restoration and/or improvement of cognitive functions in a subject in need thereof.

In other words, the invention relates to an activator of the autophagy for use in the treatment of cognitive troubles in a subject in need thereof.

In one embodiment, the autophagy is the neuronal autophagy.

In one embodiment, the cognitive functions are knowledge, attention, memory and working memory, judgment and evaluation, reasoning and computation, problem solving and decision making, comprehension and production of language.

In one embodiment, the cognitive troubles are troubles in knowledge, attention, memory and working memory, judgment and evaluation, processing speed, reasoning and computation, executive functioning, visuospatial abilities, problem solving and decision making, comprehension and production of language.

Thus, in one embodiment, the invention relates to an activator of the autophagy for use in the restoration and/or improvement of memory in a subject in need thereof.

According to the invention, the activator of the autophagy may be used in old people with age-related memory decline.

Thus, the invention also relates to an activator of the autophagy for use in the restoration and/or improvement of the age-related memory decline or age-related memory loss in a subject in need thereof.

Thus, the invention also relates to an activator of the autophagy for use in the treatment of dementia in a subject in need thereof.

In others words, the invention relates to an activator of the autophagy for use in the prevention and/or reversion of the deleterious effects of aging on cognitive function.

In one embodiment, the invention relates to an activator of the autophagy for use in the prevention and/or reversion of the deleterious effects of aging on memory.

According to the invention, the activator of the autophagy may be used in people with diseases which have an impact on the memory. Such diseases may be Alzheimer's disease, dementia, amnesia, Hyperthymestic syndrome, Huntington's disease, Parkinson's disease, Stress or Wernicke-Korsakoffs syndrome.

Thus, a second aspect of the invention relates to an activator of the autophagy to restore and/or improve the memory in a subject in need thereof suffering from Alzheimer's disease, dementia, amnesia, Hyperthymestic syndrome, Huntington's disease, Parkinson's disease, Stress or Wernicke-Korsakoffs syndrome.

As used herein, the term “autophagy” denotes a natural intracellular system that delivers cytoplasmic constituents (proteins and organelles) to the lysosome for degradation. Three forms of autophagy are commonly described: macroautophagy, microautophagy, and chaperone-mediated autophagy (CMA), along with mitophagy. The inventor focus on macroautophagy (which we will hereafter refer to as autophagy), the best-characterized autophagic mechanism in eukaryotic cells in which portions of the cytoplasm are sequestered within double- or multimembraned vesicles known as an autophagosome and then delivered to lysosomes for bulk degradation. Indeed, it is an essential proteostasis and stress response mechanism that maintains cellular health by regulating the quantity and quality of organelles and macromolecules through lysosomal degradation.

The initial phases of macroautophagy consist of the formation of a phagophore (also called isolation membrane), the engulfment of cytoplasmic material by the phagophore, the elongation of the phagophore membrane, and fusion of its edges to close the autophagosome. The outer membrane of the autophagosome fuses with the lysosome to form the autolysosome (also called autophagolysosome) in which the luminal material including the internal membrane is degraded.

The biogenesis of autophagosome is orchestrated by multiple signaling pathways and dynamic membrane complexes containing Autophagy-related (ATG) proteins, such as Beclin-1, ATG14, Vps34, that are essential for the formation of double membrane autophagosomes. The ATGS/12/16L1 complex finally allows for the lipidation of the LC3 protein (LC3II) which is then recruited to the site(s) of nascent autophagosome to favor the growing of the double membrane structure and its maturation The autophagosome eventually fuses with lysosomes and the contents are degraded and recycled.

At the cellular level, macroautophagy serves as the sole catabolic mechanism for degrading organelles and protein aggregates. Autophagy participates in regulation of intracellular component turnover, such as amino acids, lipids, and carbohydrates thereby contributing directly to cell metabolism and energy production. Indeed, autophagy control cellular homeostasis and organelle turnover. Dysregulation of autophagy is associated with cancer, diabetes, obesity and neurodegenerative diseases.

In one embodiment, the invention relates to an activator of the macroautophagy for use in the restoration and/or improvement of the memory in a subject in need thereof.

As used herein, the term “activator of autophagy” and more particularly “activator of neuronal autophagy” or “activator of hippocampal autophagy” denotes a compound which is able to restore and/or improve the macroautophagy. This kind of compound is thus able to restore and/or improve both: the adaptive mechanism of the cell in response to physiological and environmental stimuli and the capacity of the cells to maintain the quantity and quality of organelles and macromolecules through lysosomal degradation.

Activator of autophagy are well known in the art (see for example Ya-ping YANG et al 2013 and www.sigmaaldrich.com/life-science/cell-biology/cell-biology-products.html?TablePage=104899444). Activators of autophagy may be selected in the group consisting in Earle's balanced salt solution (EBSS), Brefeldin A, Thapsigargin, Tunicamycin, Rapamycin, CCI-779, RAD001, AP23576, Small molecule enhancers rapamycin (SMER), Trehalose, Lithium chloride, L-690,330, Carbamazepine, Valproic acid sodium salt, N-Acetyl-D-sphingosine (C2-ceramide), Penitrem A, Calpastatin, Xestospongin B, Akebia saponin, Amiodarone hydrochloride, ATG13, GF 109203X synthetic, GF 109203X hydrochloride, N-Hexanoyl-D-sphingosine, MRT68921 dihydrochloride, Niclosamide, Qc1, Rottlerin, STF-62247, Tamoxifen, Temsirolimus, ULK Active, Z36 and Hydroxycitrate.

According to the invention, the term “subject” denotes a mammal, such as a rodent, a feline, a canine, and a primate. Particularly, the subject according to the invention is a human. Particularly, the subject denotes an old human or a human with cognitive troubles and particularly memory troubles.

In a particular embodiment, the activator of the invention can be administrated orally, intra-nasally, parenterally, Intraocularly, intravenously, intramuscularly, intrathecally, or subcutaneously to subject in need thereof. The activator of the invention may also be administrated by hippocampal stereotactic injections.

In particular embodiment, the activator of the invention is administrated by systemic administration.

As used herein, the term “systemic administration” has its general meaning in the art and refers to a route of administration of medication into the circulatory system so that the entire body is affected.

In a particularly embodiment, the activator of the autophagy is the beclin 1 of SEQ ID NO: 1.

SEQ ID NO:1 of the beclin 1:

MEGSKTSNNS TMQVSFVCQR CSQPLKLDTS FKILDRVTIQ
ELTAPLLTTA QAKPGETQEE ETNSGEEPFI ETPRQDGVSR
RFIPPARMMS TESANSFTLI GEASDGGTME NLSRRLKVTG
DLFDIMSGQT DVDHPLCEEC TDTLLDQLDT QLNVTENECQ
NYKRCLEILE QMNEDDSEQL QMELKELALE EERLIQELED
VEKNRKIVAE NLEKVQAEAE RLDQEEAQYQ REYSEFKRQQ
LELDDELKSV ENQMRYAQTQ LDKLKKTNVF NATFHIWHSG
QFGTINNFRL GRLPSVPVEW

### NEINAAWGQT VLLLHALANK MGLKFQRYRL VPYGNHSYLE SLTDKSKELP LYCSGGLRFF WDNKFDHAMV AFLDCVQQFK EEVEKGETRF CLPYRMDVEK GKIEDTGGSG GSYSIKTQFN SEEQWTKALK FMLTNLKWGL AWVSSQFYNK

In another embodiment, the activator of the autophagy is a peptide derived from the beclin 1 protein wherein the peptide has a sequence comprising residues 270 to 278 of the amino acid sequence SEQ ID NO:1.

In another embodiment, the activator of the autophagy is a peptide derived from the beclin 1 protein wherein the peptide has a sequence comprising residues 270 to 283 of the amino acid sequence SEQ ID NO:1.

In another embodiment, the activator of the autophagy is a peptide comprising an amino acid sequence of formula (I) (SEQ ID NO: 2):

Xaa1-N-A-T-F-Xaa2-Xaa3-Xaa4-Xaa5,

wherein:

Xaa1, Xaa2-Xaa3-Xaa4, Xaa5 is the amino acids Alanine (A), Arginine (R), Asparagine (N), Aspartic acid (D), Cysteine (C), Glutamic acid (E), Glutamine (Q), Glycine (G), Histidine (H), Isoleucine (I), Leucine (L), Lysine (K), Methionine (M), Phenylalanine (F), Proline (P), Serine (S), Threonine (T), Tryptophan (W), Tyrosine (Y), Valine (V), allyl glycine (AllylGly), norleucine, norvaline, biphenylalanine (Bip), citrulline (Cit), 4-guanidinophenylalanine (Phe(Gu)), homoarginine (hArg), homolysine (hLys), 2-naphtylalanine (2-Nal), ornithine (Orn) or pentafluorophenylalanine.

In another embodiment, the invention relates to a peptide consisting in an amino acid sequence of formula (I) (SEQ ID NO: 2):

Xaa1-N-A-T-F-Xaa2-Xaa3-Xaa4-Xaa5,

wherein:

Xaa1, Xaa2-Xaa3-Xaa4, Xaa5 is the amino acids Alanine (A), Arginine (R), Asparagine (N), Aspartic acid (D), Cysteine (C), Glutamic acid (E), Glutamine (Q), Glycine (G), Histidine (H), Isoleucine (I), Leucine (L), Lysine (K), Methionine (M), Phenylalanine (F), Proline (P), Serine (S), Threonine (T), Tryptophan (W), Tyrosine (Y), Valine (V), allyl glycine (AllylGly), norleucine, norvaline, biphenylalanine (Bip), citrulline (Cit), 4-guanidinophenylalanine (Phe(Gu)), homoarginine (hArg), homolysine (hLys), 2-naphtylalanine (2-Nal), ornithine (Orn) or pentafluorophenylalanine.

In a particular embodiment, the activator of the autophagy is a peptide comprising or consisting in an amino acid sequences: FNATFHIWH (SEQ ID NO: 3), VFNATFEIWHD (SEQ ID NO: 4), CFNATFEIWHD (SEQ ID NO: 5), VWNATFEIWHD (SEQ ID NO: 6), VFNATFDIWHD (SEQ ID NO: 7), VFNATFELWHD (SEQ ID NO: 8), VFNATFEIFHD (SEQ ID NO: 9), VFNATFEIWYD (SEQ ID NO: 10), VFNATFEIWHE (SEQ ID NO: 11), VWNATFELWHD (SEQ ID NO: 12), VFNATFEVWHD (SEQ ID NO: 13), VLNATFEIWHD (SEQ ID NO: 14), VFNATFEMWHD (SEQ ID NO: 15), VWNATFHIWHD (SEQ ID NO: 16), VFNATFEFWHD (SEQ ID NO: 17), VFNATFEYWHD (SEQ ID NO: 18), VFNATFERWHD (SEQ ID NO: 19), FNATFEIWHD (SEQ ID NO: 20), VFNATFEIWH (SEQ ID NO: 21), FNATFEIWH (SEQ ID NO: 22), WNATFHIWH (SEQ ID NO: 23), VWNATFHIWH (SEQ ID NO: 24) or WNATFHIWHD (SEQ ID NO: 25), or a function-conservative variant thereof.

In one embodiment, the activator of the autophagy is a peptide comprising an amino acid sequence of formula (II) (SEQ ID NO: 26): V-F-N-A-T-F-Xaa1-I-W-H-Xaa2-G-Xaa3-F-G wherein Xaa1 may be Glutamic acid (E) or Histidine (H), Xaa2 may Aspartic acid (D) or Serine (S) and Xaa3 may be Glutamine (Q) or Glutamic acid (E).

In one embodiment, the activator of autophagy is a peptide comprising an amino acids sequence: VFNATFEIWHDGEFG (SEQ ID NO: 27) or VFNATFHIWHSGQFG (SEQ ID NO: 28) or a function-conservative variant thereof.

In one embodiment, the activator of autophagy is a peptide consisting of the amino acids sequence: VFNATFEIWHDGEFG (SEQ ID NO: 27) or VFNATFHIWHSGQFG (SEQ ID NO: 28) or a function-conservative variant thereof.

In one embodiment, the activator of the autophagy is a peptide comprising an amino acid sequence of formula (III) (SEQ ID NO: 29): T-N-V-F-N-A-T-F-Xaa1-I-W-H-Xaa2-G-Xaa3-F-G-T wherein Xaa1 may be Glutamic acid (E) or Histidine (H), Xaa2 may Aspartic acid (D) or Serine (S) and Xaa3 may be Glutamine (Q) or Glutamic acid (E).

In one embodiment, the activator of autophagy is a peptide comprising an amino acids sequence: TNVFNATFEIWHDGEFGT (SEQ ID NO: 30) or TNVFNATFHIWHSGQFGT (SEQ ID NO: 31) or a function-conservative variant thereof.

In one embodiment, the activator of autophagy is a peptide consisting of the amino acids sequence: TNVFNATFEIWHDGEFGT (SEQ ID NO: 30) or TNVFNATFHIWHSGQFGT (SEQ ID NO: 31) or a function-conservative variant thereof.

According to the invention the amino acids of the invention may be left (s) or right (r) in zwitterionic form.

According to the invention the amino acids of the invention may be in dextrorotation or levorotation.

A further aspect of the present invention relates to a fusion protein comprising a peptide according to the invention (which is an activator of the autophagy) that is fused to at least one heterologous polypeptide.

The term “fusion protein” refers to the polypeptide according to the invention that is fused directly or via a spacer to at least one heterologous polypeptide.

According to the invention, the fusion protein comprises the polypeptide according to the invention that is fused either directly or via a spacer at its C-terminal end to the N-terminal end of the heterologous polypeptide, or at its N-terminal end to the C-terminal end of the heterologous polypeptide.

As used herein, the term “directly” means that the (first or last) amino acid at the terminal end (N or C-terminal end) of the polypeptide is fused to the (first or last) amino acid at the terminal end (N or C-terminal end) of the heterologous polypeptide.

In other words, in this embodiment, the last amino acid of the C-terminal end of said peptide is directly linked by a covalent bond to the first amino acid of the N-terminal end of said heterologous polypeptide, or the first amino acid of the N-terminal end of said peptide is directly linked by a covalent bond to the last amino acid of the C-terminal end of said heterologous polypeptide.

As used herein, the term “spacer” refers to a sequence of at least one amino acid that links the peptide of the invention to the heterologous polypeptide. Such a spacer may be useful to prevent steric hindrances. According to the invention, the polypeptide may be coupled to the peptide through linkers or spacers known in the art, such as polyglycine, ε-aminocaproic, etc.

In some embodiments, the heterologous polypeptide is a cell-penetrating peptide, a Transactivator of Transcription (TAT) cell penetrating sequence, a cell permeable peptide or a membranous penetrating sequence.

The term “cell-penetrating peptides” are well known in the art and refers to cell permeable sequence or membranous penetrating sequence such as protein-derived (e.g. tat, smac, pen, pVEC, bPrPp, PIsl, VP22, M918, pep-3); chimeric (e.g. TP, TP10, MPGA) or synthetic (e.g. MAP, Pep-1, Oligo Arg) cell-penetrating peptides (see, e.g. “Peptides as Drugs: Discovery and Development”, Ed. Bernd Groner, 2009 WILEY-VCH Verlag GmbH & Co, KGaA, Weinheim, esp. Chap 7: “The Internalization Mechanisms and Bioactivity of the Cell-Penetrating Peptides”, Mats Hansen, Elo Eriste, and Ulo Langel, pp. 125-144) or penetratin, TAT mitochondrial penetrating sequence and compounds (Bechara and Sagan, 2013; Jones and Sayers, 2012; Khafagy el and Morishita, 2012; Malhi and Murthy, 2012). According to the invention, the cell-penetratin peptide can be the RGD-4C (CCDCRGDCFC; SEQ ID NO:32), NGR (CCNGRC; SEQ ID NO:33), CREKA, LyP-1 (CGNKRTRGC; SEQ ID NO:34), F3, SMS (SMSIARL; SEQ ID NO:35), IF7, and H2009.1 (Li et al. Bioorg Med Chem. 2011 Sep. 15; 19(18):5480-9).

In a particular embodiment, the heterologous polypeptide is an internalization sequence derived either from the homeodomain of Drosophila Antennapedia/Penetratin (Antp) protein or the Transactivator of Transcription (TAT) cell penetrating sequence of SEQ ID NO: YGRKKRRQRRR (SEQ ID NO:36).

In a particular embodiment, one, two or three glycine (G) residue are added at the C-terminal end of the TAT cell penetrating sequences (SEQ ID NO:36).

In a particular embodiment, the TAT peptide is fused directly or via a spacer of 1, 2 or 3 glycine (G) to the peptide comprising an amino acid sequence of formula (I), (II) or (III).

In another embodiment, the peptide of the invention is the TAT-Beclin 1 of SEQ ID NO:37 (a peptide derived form the beclin and fused to the cell-penetrating peptide TAT).

SEQ ID NO:37 of the TAT-beclin 1:

YGRKKRRQRRRGGTNVFNATFEIWHDGEFGT

In another particular embodiment, the invention relates to a peptide comprising the amino acids sequence: YGRKKRRQRRRGGTNVFNATFEIWHDGEFGT (SEQ ID NO:37) or a function-conservative variant thereof to restore and/or improve cognitive functions in a subject in need thereof.

In other words, the invention relates to a peptide comprising the amino acids sequence: YGRKKRRQRRRGGTNVFNATFEIWHDGEFGT (SEQ ID NO:37) or a function-conservative variant thereof for use in the treatment of cognitive troubles in a subject in need thereof.

In another particular embodiment, the invention relates to a peptide consisting to the amino acids sequence: YGRKKRRQRRRGGTNVFNATFEIWHDGEFGT (SEQ ID NO:37) or a function-conservative variant thereof to restore and/or improve cognitive functions in a subject in need thereof.

In other words, the invention relates to a peptide consisting to the amino acids sequence: YGRKKRRQRRRGGTNVFNATFEIWHDGEFGT (SEQ ID NO:37) or a function-conservative variant thereof for use in the treatment of cognitive troubles in a subject in need thereof.

In another embodiment, the peptides of the invention may differ from 1, 2 or 3 amino acids to the formula (I), (II) or (III).

In one embodiment, the peptide of the invention comprises at least 60%, 65%, 70%, 75%, 80%, 85%, 90%, 95%, 96%, 97%, 98% or 99% of identity over the peptides of formula (I), (II) or (III), and is still able to induce autophagy.

In one embodiment, the peptide of the invention consists of at least 60%, 65%, 70%, 75%, 80%, 85%, 90%, 95%, 96%, 97%, 98% or 99% of identity over the peptides of formula (I), (II) or (III), and is still able to induce autophagy.

In another embodiment, the peptide of SEQ ID NO:37 according to the invention may differ from 1, 2 or 3 amino acids to the SEQ ID NO: 9.

In one embodiment, the peptide of the invention comprises at least 60%, 65%, 70%, 75%, 80%, 85%, 90%, 95%, 96%, 97%, 98% or 99% of identity over said SEQ ID NO: 37, and is still able to induce autophagy.

In one embodiment, the peptide of the invention consists of at least 60%, 65%, 70%, 75%, 80%, 85%, 90%, 95%, 96%, 97%, 98% or 99% of identity over said SEQ ID NO: 37, and is still able to induce autophagy.

To verify whether newly generated peptides are still able to induce autophagy, first an evaluation of the autophagy formation and flux in primary hippocampal neuronal cultures and after local/systemic injections in mouse hippocampus will be performed. Second, the functional activity of the positive peptides will be tested in young and old mice after local/systemic injections by performing Object Location Memory (OLM) task assays (a simple, fast and robust analysis of the hippocampal functional activity). In the advent of positive results, the active component will be then evaluated with respect to the whole battery of behavioral, assessing memory-related behavior (Novel object recognition, (NOR), contextual fear conditioning (CFC), Morris Water maze task (MWM)), cellular (induction of dendritic spine formation) and molecular aspects (molecular strength of the synaptic activity).

In one embodiment of the invention, the peptide of the invention is an amino acid sequence of less than 50 amino acids long that comprises the amino acid sequence of formula (I), (II), (III) or SEQ ID NO:37 as defined here above.

In one embodiment of the invention, the peptide of the invention is an amino acid sequence of less than 45 amino acids long that comprises the amino acid sequence of formula (I), (II), (III) or SEQ ID NO:37 as defined here above.

In one embodiment of the invention, the peptide of the invention is an amino acid sequence of less than 40 amino acids long that comprises the amino acid sequence of formula (I), (II), (III) or SEQ ID NO:37 as defined here above.

In one embodiment of the invention, the peptide of the invention is an amino acid sequence of less than 35 amino acids long that comprises the amino acid sequence of formula (I), (II), (III) or SEQ ID NO:37 as defined here above.

As used herein, the term “Function-conservative variants” refer to those in which a given amino acid residue in a protein or enzyme has been changed (inserted, deleted or substituted) without altering the overall conformation and function of the peptide. Such variants include protein having amino acid alterations such as deletions, insertions and/or substitutions. A “deletion” refers to the absence of one or more amino acids in the protein. An “insertion” refers to the addition of one or more of amino acids in the protein. A “substitution” refers to the replacement of one or more amino acids by another amino acid residue in the protein. Typically, a given amino acid is replaced by an amino acid having similar properties (such as, for example, polarity, hydrogen bonding potential, acidic, basic, hydrophobic, aromatic, and the like). This given amino acid can be a natural amino acid or a non natural amino acid. Amino acids other than those indicated as conserved may differ in a protein so that the percent protein or amino acid sequence similarity between any two proteins of similar function may vary and may be, for example, from 70% to 99% as determined according to an alignment scheme such as by the Cluster Method, wherein similarity is based on the MEGALIGN algorithm. A “function-conservative variant” also includes a polypeptide which has at least 60% amino acid identity as determined by BLAST or FASTA algorithms, preferably at least 75%, more preferably at least 85%, still preferably at least 90%, and even more preferably at least 95%, and which has the same or substantially similar properties or functions as the native or parent protein to which it is compared. Two amino acid sequences are “substantially homologous” or “substantially similar” when greater than 80%, preferably greater than 85%, preferably greater than 90% of the amino acids are identical, or greater than about 90%, preferably greater than 95%, are similar (functionally identical) over the whole length of the shorter sequence. Preferably, the similar or homologous sequences are identified by alignment using, for example, the GCG (Genetics Computer Group, Program Manual for the GCG Package, Version 7, Madison, Wis.) pileup program, or any of sequence comparison algorithms such as BLAST, FASTA, etc.

Examples of conservative substitutions include the substitution of one non-polar (hydrophobic) residue such as isoleucine, valine, leucine or methionine for another, the substitution of one polar (hydrophilic) residue for another such as between arginine and lysine, between glutamine and asparagine, between glycine and serine, the substitution of one basic residue such as lysine, arginine or histidine for another, or the substitution of one acidic residue, such as aspartic acid or glutamic acid or another.

The term “conservative substitution” also includes the use of a chemically derivatized residue in place of a non-derivatized residue. “Chemical derivative” refers to a subject peptide having one or more residues chemically derivatized by reaction of a functional side group.

Examples of such derivatized molecules include for example, those molecules in which free amino groups have been derivatized to form amine hydrochlorides, p-toluene sulfonyl groups, carbobenzoxy groups, t-butyloxycarbonyl groups, chloroacetyl groups or formyl groups. Free carboxyl groups may be derivatized to form salts, methyl and ethyl esters or other types of esters or hydrazides. Free hydroxyl groups may be derivatized to form O-acyl or O-alkyl derivatives. The imidazole nitrogen of histidine may be derivatized to form N-im-benzylhistidine. Chemical derivatives also include peptides that contain one or more naturally-occurring amino acid derivatives of the twenty standard amino acids. For examples: 4-hydroxyproline may be substituted for proline; 5-hydroxylysine may be substituted for lysine; 3-methylhistidine may be substituted for histidine; homoserine may be substituted for serine; and ornithine may be substituted for lysine. The term “conservative substitution” also includes the use of non natural amino acids aimed to control and stabilize peptides or proteins secondary structures. These non natural amino acids are chemically modified amino acids such as prolinoamino acids, beta-amino acids, N-methylamino acids, cyclopropylamino acids, alpha, alpha-substituted amino acids as describe here below. These non natural amino acids may include also fluorinated, chlorinated, brominated- or iodinated modified amino acids.

In one embodiment, peptides of the invention may comprise a tag. A tag is an epitope-containing sequence which can be useful for the purification of the peptides. It is attached to by a variety of techniques such as affinity chromatography, for the localization of said peptide or polypeptide within a cell or a tissue sample using immuno labeling techniques, the detection of said peptide or polypeptide by immunoblotting etc. Examples of tags commonly employed in the art are the GST (glutathion-S-transferase)-tag, the FLAG™-tag, the Strep-tag™, V5 tag, myc tag, His tag etc.

In one embodiment, peptides of the invention may be labelled by a fluorescent dye. Dye-labelled fluorescent peptides are important tools in cellular studies. Peptides can be labelled on the N-terminal side or on the C-terminal side.

N-Terminal Peptide Labeling Using Amine-Reactive Fluorescent Dyes:

Amine-reactive fluorescent probes are widely used to modify peptides at the N-terminal or lysine residue. A number of fluorescent amino-reactive dyes have been developed to label various peptides, and the resultant conjugates are widely used in biological applications. Three major classes of amine-reactive fluorescent reagents are currently used to label peptides: succinimidyl esters (SE), isothiocyanates and sulfonyl chlorides.

C-Terminal Labeling Using Amine-Containing Fluorescent Dyes:

Amine-containing dyes are used to modify peptides using water-soluble carbodiimides (such as EDC) to convert the carboxy groups of the peptides into amide groups. Either NHS or NHSS may be used to improve the coupling efficiency of EDC-mediated protein-carboxylic acid conjugations.

In specific embodiments, it is contemplated that peptides used in the therapeutic methods of the present invention may be modified in order to improve their therapeutic efficacy. Such modification of therapeutic compounds may be used to decrease toxicity, increase circulatory time, or modify biodistribution. For example, the toxicity of potentially important therapeutic compounds can be decreased significantly by combination with a variety of drug carrier vehicles that modify biodistribution.

A strategy for improving drug viability is the utilization of water-soluble polymers. Various water-soluble polymers have been shown to modify biodistribution, improve the mode of cellular uptake, change the permeability through physiological barriers; and modify the rate of clearance from the body. To achieve either a targeting or sustained-release effect, water-soluble polymers have been synthesized that contain drug moieties as terminal groups, as part of the backbone, or as pendent groups on the polymer chain.

Polyethylene glycol (PEG) has been widely used as a drug carrier, given its high degree of biocompatibility and ease of modification. Attachment to various drugs, proteins, and liposomes has been shown to improve residence time and decrease toxicity. PEG can be coupled to active agents through the hydroxyl groups at the ends of the chain and via other chemical methods; however, PEG itself is limited to at most two active agents per molecule. In a different approach, copolymers of PEG and amino acids were explored as novel biomaterials which would retain the biocompatibility properties of PEG, but which would have the added advantage of numerous attachment points per molecule (providing greater drug loading), and which could be synthetically designed to suit a variety of applications.

Those of skill in the art are aware of PEGylation techniques for the effective modification of drugs. For example, drug delivery polymers that consist of alternating polymers of PEG and tri-functional monomers such as lysine have been used by VectraMed (Plainsboro, N.J.). The PEG chains (typically 2000 daltons or less) are linked to the a- and e-amino groups of lysine through stable urethane linkages. Such copolymers retain the desirable properties of PEG, while providing reactive pendent groups (the carboxylic acid groups of lysine) at strictly controlled and predetermined intervals along the polymer chain. The reactive pendent groups can be used for derivatization, cross-linking, or conjugation with other molecules. These polymers are useful in producing stable, long-circulating pro-drugs by varying the molecular weight of the polymer, the molecular weight of the PEG segments, and the cleavable linkage between the drug and the polymer. The molecular weight of the PEG segments affects the spacing of the drug/linking group complex and the amount of drug per molecular weight of conjugate (smaller PEG segments provides greater drug loading). In general, increasing the overall molecular weight of the block co-polymer conjugate will increase the circulatory half-life of the conjugate. Nevertheless, the conjugate must either be readily degradable or have a molecular weight below the threshold-limiting glomular filtration (e.g., less than 45 kDa).

In addition, to the polymer backbone being important in maintaining circulatory half-life, and biodistribution, linkers may be used to maintain the therapeutic agent in a pro-drug form until released from the backbone polymer by a specific trigger, typically enzyme activity in the targeted tissue. For example, this type of tissue activated drug delivery is particularly useful where delivery to a specific site of biodistribution is required and the therapeutic agent is released at or near the site of pathology. Linking group libraries for use in activated drug delivery are known to those of skill in the art and may be based on enzyme kinetics, prevalence of active enzyme, and cleavage specificity of the selected disease-specific enzymes (see e.g., technologies of established by VectraMed, Plainsboro, N.J.). Such linkers may be used in modifying the peptides-derived described herein for therapeutic delivery.

According to the invention, peptides may be produced by conventional automated peptide synthesis methods or by recombinant expression. General principles for designing and making proteins are well known to those of skill in the art.

Peptides of the invention may be synthesized in solution or on a solid support in accordance with conventional techniques. Various automatic synthesizers are commercially available and can be used in accordance with known protocols as described in Stewart and Young; Tam et al., 1983; Merrifield, 1986 and Barany and Merrifield, Gross and Meienhofer, 1979. Peptides of the invention may also be synthesized by solid-phase technology employing an exemplary peptide synthesizer such as a Model 433A from Applied Biosystems Inc. The purity of any given protein; generated through automated peptide synthesis or through recombinant methods may be determined using reverse phase HPLC analysis. Chemical authenticity of each peptide may be established by any method well known to those of skill in the art.

As an alternative to automated peptide synthesis, recombinant DNA technology may be employed wherein a nucleotide sequence which encodes a protein of choice is inserted into an expression vector, transformed or transfected into an appropriate host cell and cultivated under conditions suitable for expression as described herein below. Recombinant methods are especially preferred for producing longer polypeptides.

A variety of expression vector/host systems may be utilized to contain and express the peptide or protein coding sequence. These include but are not limited to microorganisms such as bacteria transformed with recombinant bacteriophage, plasmid or cosmid DNA expression vectors; yeast transformed with yeast expression vectors (Giga-Hama et al., 1999); insect cell systems infected with virus expression vectors (e.g., baculovirus, see Ghosh et al., 2002); plant cell systems transfected with virus expression vectors (e.g., cauliflower mosaic virus, CaMV; tobacco mosaic virus, TMV) or transformed with bacterial expression vectors (e.g., Ti or pBR322 plasmid; see e.g., Babe et al., 2000); or animal cell systems. Those of skill in the art are aware of various techniques for optimizing mammalian expression of proteins, see e.g., Kaufman, 2000; Colosimo et al., 2000. Mammalian cells that are useful in recombinant protein productions include but are not limited to VERO cells, HeLa cells, Chinese hamster ovary (CHO) cell lines, COS cells (such as COS-7), W138, BHK, HepG2, 3T3, RIN, MDCK, A549, PC12, K562 and 293 cells. Exemplary protocols for the recombinant expression of the peptide substrates or fusion polypeptides in bacteria, yeast and other invertebrates are known to those of skill in the art and a briefly described herein below. U.S. Pat. Nos. 6,569,645; 6,043,344; 6,074,849; and 6,579,520 provide specific examples for the recombinant production of peptides and these patents are expressly incorporated herein by reference for those teachings. Mammalian host systems for the expression of recombinant proteins also are well known to those of skill in the art. Host cell strains may be chosen for a particular ability to process the expressed protein or produce certain post-translation modifications that will be useful in providing protein activity. Such modifications of the polypeptide include, but are not limited to, acetylation, carboxylation, glycosylation, phosphorylation, lipidation and acylation. Post-translational processing which cleaves a “prepro” form of the protein may also be important for correct insertion, folding and/or function. Different host cells such as CHO, HeLa, MDCK, 293, WI38, and the like have specific cellular machinery and characteristic mechanisms for such post-translational activities and may be chosen to ensure the correct modification and processing of the introduced, foreign protein.

In the recombinant production of the peptides-derived of the invention, it would be necessary to employ vectors comprising polynucleotide molecules for encoding the peptides-derived. Methods of preparing such vectors as well as producing host cells transformed with such vectors are well known to those skilled in the art. The polynucleotide molecules used in such an endeavor may be joined to a vector, which generally includes a selectable marker and an origin of replication, for propagation in a host. These elements of the expression constructs are well known to those of skill in the art. Generally, the expression vectors include DNA encoding the given protein being operably linked to suitable transcriptional or translational regulatory sequences, such as those derived from a mammalian, microbial, viral, or insect genes. Examples of regulatory sequences include transcriptional promoters, operators, or enhancers, mRNA ribosomal binding sites, and appropriate sequences which control transcription and translation.

The terms “expression vector,” “expression construct” or “expression cassette” are used interchangeably throughout this specification and are meant to include any type of genetic construct containing a nucleic acid coding for a gene product in which part or all of the nucleic acid encoding sequence is capable of being transcribed.

The choice of a suitable expression vector for expression of the peptides or polypeptides of the invention will of course depend upon the specific host cell to be used, and is within the skill of the ordinary artisan. Methods for the construction of mammalian expression vectors are disclosed, for example, in Okayama and Berg, 1983; Cosman et al., 1986; Cosman et al., 1984; EP-A-0367566; and WO 91/18982. Other considerations for producing expression vectors are detailed in e.g., Makrides et al., 1999; Kost et al., 1999. Wurm et al., 1999 is incorporated herein as teaching factors for consideration in the large-scale transient expression in mammalian cells for recombinant protein production.

Expression requires that appropriate signals be provided in the vectors, such as enhancers/promoters from both viral and mammalian sources that may be used to drive expression of the nucleic acids of interest in host cells. Usually, the nucleic acid being expressed is under transcriptional control of a promoter. A “promoter” refers to a DNA sequence recognized by the synthetic machinery of the cell, or introduced synthetic machinery, required to initiate the specific transcription of a gene. Nucleotide sequences are operably linked when the regulatory sequence functionally relates to the DNA encoding the peptide of interest (i.e., 4N1K, a variant and the like). Thus, a promoter nucleotide sequence is operably linked to a given DNA sequence if the promoter nucleotide sequence directs the transcription of the sequence.

Similarly, the phrase “under transcriptional control” means that the promoter is in the correct location and orientation in relation to the nucleic acid to control RNA polymerase initiation and expression of the gene. Any promoter that will drive the expression of the nucleic acid may be used. The particular promoter employed to control the expression of a nucleic acid sequence of interest is not believed to be important, so long as it is capable of directing the expression of the nucleic acid in the targeted cell. Thus, where a human cell is targeted, it is preferable to position the nucleic acid coding region adjacent to and under the control of a promoter that is capable of being expressed in a human cell. Generally speaking, such a promoter might include either a human or viral promoter. Common promoters include, e.g., the human cytomegalovirus (CMV) immediate early gene promoter, the SV40 early promoter, the Rous sarcoma virus long terminal repeat, [beta]-actin, rat insulin promoter, the phosphoglycerol kinase promoter and glyceraldehyde-3-phosphate dehydrogenase promoter, all of which are promoters well known and readily available to those of skill in the art, can be used to obtain high-level expression of the coding sequence of interest. The use of other viral or mammalian cellular or bacterial phage promoters which are well-known in the art to achieve expression of a coding sequence of interest is contemplated as well, provided that the levels of expression are sufficient to produce a recoverable yield of protein of interest. By employing a promoter with well-known properties, the level and pattern of expression of the protein of interest following transfection or transformation can be optimized. Inducible promoters also may be used.

Another regulatory element that is used in protein expression is an enhancer. These are genetic elements that increase transcription from a promoter located at a distant position on the same molecule of DNA. Where an expression construct employs a cDNA insert, one will typically desire to include a polyadenylation signal sequence to effect proper polyadenylation of the gene transcript. Any polyadenylation signal sequence recognized by cells of the selected transgenic animal species is suitable for the practice of the invention, such as human or bovine growth hormone and SV40 polyadenylation signals.

Another object of the invention relates to a nucleic acid encoding an amino acids sequence of formula (I), (II), (III) or SEQ ID NO:37 or a function-conservative variant thereof as described here above for use in the restoration and/or improvement of cognitive functions in a subject in need thereof.

Another object of the invention relates to a nucleic acid encoding an amino acids sequence of formula (I), (II), (III) or SEQ ID NO:37 or a function-conservative variant thereof as described here above for use in the treatment of cognitive troubles in a subject in need thereof.

In one embodiment, said nucleic acid encoding an amino acids sequence consisting on SEQ ID NO:37.

Nucleic acids of the invention may be produced by any technique known per se in the art, such as, without limitation, any chemical, biological, genetic or enzymatic technique, either alone or in combination(s).

Another object of the invention is an expression vector comprising a nucleic acid sequence encoding an amino sequence of formula (I), (II), (III) or SEQ ID NO:37 or a function-conservative variant thereof as described here above to restore and/or improve cognitive functions in a subject in need thereof.

Another object of the invention is an expression vector comprising a nucleic acid sequence encoding an amino sequence comprising of formula (I), (II), (III) or SEQ ID NO:37 or a function-conservative variant thereof as described here above for use in the treatment of cognitive troubles in a subject in need thereof.

According to the invention, expression vectors suitable for use in the invention may comprise at least one expression control element operationally linked to the nucleic acid sequence. The expression control elements are inserted in the vector to control and regulate the expression of the nucleic acid sequence. Examples of expression control elements include, but are not limited to, lac system, operator and promoter regions of phage lambda, yeast promoters and promoters derived from polyoma, adenovirus, retrovirus, lentivirus or SV40. Additional preferred or required operational elements include, but are not limited to, leader sequence, termination codons, polyadenylation signals and any other sequences necessary or preferred for the appropriate transcription and subsequent translation of the nucleic acid sequence in the host system. It will be understood by one skilled in the art that the correct combination of required or preferred expression control elements will depend on the host system chosen. It will further be understood that the expression vector should contain additional elements necessary for the transfer and subsequent replication of the expression vector containing the nucleic acid sequence in the host system. Examples of such elements include, but are not limited to, origins of replication and selectable markers. It will further be understood by one skilled in the art that such vectors are easily constructed using conventional methods or commercially available.

Another object of the invention is a host cell comprising an expression vector as described here above

According to the invention, examples of host cells that may be used are eukaryote cells, such as animal, plant, insect and yeast cells and prokaryotes cells, such as E. coli. The means by which the vector carrying the gene may be introduced into the cells include, but are not limited to, microinjection, electroporation, transduction, or transfection using DEAE-dextran, lipofection, calcium phosphate or other procedures known to one skilled in the art.

In another embodiment, eukaryotic expression vectors that function in eukaryotic cells are used. Examples of such vectors include, but are not limited to, viral vectors such as retrovirus, adenovirus, adeno-associated virus, herpes virus, vaccinia virus, poxvirus, poliovirus; lentivirus, bacterial expression vectors, plasmids, such as pcDNA3 or the baculovirus transfer vectors. Preferred eukaryotic cell lines include, but are not limited to, COS cells, CHO cells, HeLa cells, NIH/3T3 cells, 293 cells (ATCC #CRL1573), T2 cells, dendritic cells, or monocytes.

The invention also relates to a method for restoring and/or improving cognitive functions in a subject in need thereof comprising administering to the subject in need thereof a therapeutically effective amount of an activator of the autophagy.

The invention also relates to a method for treating cognitive troubles in a subject in need thereof comprising administering to the subject in need thereof a therapeutically effective amount of an activator of the autophagy.

As used herein, the term “treatment” or “treat” refer to both prophylactic or preventive treatment as well as curative or disease modifying treatment, including treatment of subjects at risk of contracting the disease or suspected to have contracted the disease as well as subjects who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a subject during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a subject during treatment of an illness, e.g., to keep the subject in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., disease manifestation, etc.]).

### Therapeutic Composition

Another object of the invention relates to a therapeutic composition comprising an activator of the autophagy for use in the restoration and/or improvement of cognitive functions in a subject in need thereof.

In a particularly embodiment, the invention relates to a therapeutic composition comprising an activator of the autophagy for use in the restoration and/or improvement of memory in a subject in need thereof.

Any therapeutic agent of the invention may be combined with pharmaceutically acceptable excipients, and optionally sustained-release matrices, such as biodegradable polymers, to form therapeutic compositions.

“Pharmaceutically” or “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

The form of the pharmaceutical compositions, the route of administration, the dosage and the regimen naturally depend upon the condition to be treated, the severity of the illness, the age, weight, and sex of the patient, etc.

The pharmaceutical compositions of the invention can be formulated for a topical, oral, intranasal, parenteral, intraocular, intravenous, intramuscular, hippocampal stereotactic or subcutaneous administration and the like.

In particular embodiment, the pharmaceutical compositions of the invention is formulated for systemic administration.

Preferably, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

The doses used for the administration can be adapted as a function of various parameters, and in particular as a function of the mode of administration used, of the relevant pathology, or alternatively of the desired duration of treatment.

In addition, other pharmaceutically acceptable forms include, e.g. tablets or other solids for oral administration; time release capsules; and any other form currently can be used.

Pharmaceutical compositions of the present invention may comprise a further therapeutic active agent. Further agent may selected in the group consisting in Spermidine, Hydroxycitrate, osteocalcin (see for example the patent application WO2014152497) and Resveratrol.

The invention will be further illustrated by the following figures and examples.

However, these examples and figures should not be interpreted in any way as limiting the scope of the present invention.

### FIGURES

FIG. 1: Acute modulation of hippocampal autophagy influences novel memory acquisition. A) LC3-I and LC3-II accumulation (Western blot) in 3 month-old mouse hippocampus 12 hours after stereotactic injections of pharmacological autophagy modulators: TAT-Beclin 1 or TAT-Scramble. The quantification is relative to the corresponding vehicle-treated group. B) NOR performed in 3 month-old mice after stereotactic injections with either vehicle TAT-Beclin 1 or TAT-Scramble mice. Preference index (Exploration time for the novel object/Total exploration) and discrimination index ([Exploration time for the novel object−Exploration time for the familiar object]/Total exploration) were measured for each group during the testing phase. C) CFC performed in 3 month-old mice after stereotactic injections with either vehicle, TAT-Beclin 1 or TAT-Scramble mice. Percent freezing was measured for the last minute of the training phase and the 4 min of the testing phase. All behavioral tests represented here were performed on at least two independent experiments (for each experiment: n≥5 mice per group). Data are expressed as mean±SEM. One-way ANOVA followed by Student's t test was used. *P≤0.05, **P≤0.01, ***P≤0.001, NS: not significant

FIG. 2: Maintenance of hippocampal autophagy level is necessary to counteract normal age-related memory decline and to mediate the beneficial effects of young circulating factors. A) VPS34, Beclin 1, ATGS, LC3-I and LC3-II accumulation (Western blot) and Vps34, Becn 1 and Atg5 relative expression (qPCR). B) LC3-I and LC3-II accumulation (Western blot) twelve hours after hippocampal stereotactic injections of either vehicle or TAT-Beclin 1 (1 μg/hemisphere) in 16 month- and 3 month-old mice. TAT-Beclin 1 restored LC3-II levels in old (16 months) to the level of young (3 months) mice. β-Actin was used as a loading control for each sample. C) Object location memory (OLM) task performed in 3 month- and 16-month old mice 12 hours after hippocampal stereotactic injections with either vehicle, TAT-Scramble (1 μg/hemisphere) or TAT-Beclin 1 (1 μg/hemisphere). Discrimination index ([time spent with newly located object−familiar located object]/Total exploration) was measured for each group during the testing phase. D) Novel object recognition (NOR) performed in 3 month- and 16 month-old mice 12 hours after hippocampal stereotactic injections with either vehicle, TAT-Scramble (1 μg/hemisphere) or TAT-Beclin 1 (1 μg/hemisphere). Discrimination index ([time spent with newly located object−familiar located object]/Total exploration) was measured for each group during the testing phase. E) Contextual fear conditioning (CFC) performed in 3 month- and 16 month-old mice 12 hours after hippocampal stereotactic injections with either vehicle, TAT-Scramble (1 μg/hemisphere) or TAT-Beclin 1 (1 μg/hemisphere). Percent freezing was measured over 4 min of the testing phase for each group. Discrimination index ([time spent with newly located object−familiar located object]/Total exploration) was measured for each group during the testing phase. All behavioral tests represented here were performed on at least two independent experiments (for each experiment: n≥5 mice per group). Data are expressed as mean±SEM. One-way ANOVA followed by Student's t test was used. *P≤0.05, **P≤0.01, ***P≤0.001, NS: not significant.

FIG. 3: Daily systemic injection of TAT-Beclin 1 allow to counteract normal age-related memory decline. A) Experimental protocol for behavioural test performed in mice after daily injection of TAT-Beclin or TAT-Scramble B) Contextual fear conditioning (CFC) performed in 3 month- and 16 month-old mice 14 days after daily intraperitoneal injections of either vehicle, TAT-Scramble (450 μg) or TAT-Beclin 1 (450 μg). Percent freezing was measured over 4 min of the testing phase for each group. Discrimination index ([time spent with newly located object−familiar located object]/Total exploration) was measured for each group during the testing phase. C) Novel object recognition (NOR) performed in 3 month- and 16 month-old mice 14 days after daily intraperitoneal injections of either vehicle, TAT-Scramble (450 μg) or TAT-Beclin 1 (450 μg). Preference index (Exploration time for the novel object/Total exploration) and discrimination index ([time spent with newly located object−familiar located object]/Total exploration) was measured for each group during the testing phase. All behavioral tests represented here were performed on at least two independent experiments (for each experiment: n≥5 mice per group). Data are expressed as mean±SEM. One-way ANOVA followed by Student's t test was used. *P≤0.05, **P≤0.01, ***P≤0.001, NS: not significant.

### EXAMPLE

Material & Methods

Animals

All experiments were performed on C57BL/6J male mice obtained from Janvier Laboratory stock. All mice were 3 month- or 16-months old at the start of experiments. For all experiments, we used littermates as controls. Upon arrival, mice were housed at least 2 weeks before any behavioral or molecular testing. Mice were housed five animals per cage in polycarbonate cages (35.5×18×12.5 cm), under a 12 hours light/dark cycle (lights off at 8:00 pm) with ad libitum access to food and water prior to experimentation. All behavioral experiments were performed between 10 AM and 5 PM. Group sizes were determined after performing a power calculation to lead to an 80% chance of detecting a significant difference (P≤0.05). All efforts were made to minimize animal suffering and the number of animals used accordingly to the 3R's rule. In all experiments, animals were randomly assigned to treatment groups. All behavioral experiments were performed in accordance with the European Communities for Experimental animal use (2010/63/EU) and local ethical committee review procedures and protocols.

Stereotaxic Surgery

Mice were anesthetized by intra-peritoneal injection of ketamine hydrochloride (20 mg/ml BW) (1000 Virbac) and xylazine (100 mg/ml BW) (Rompun 2%; Bayer) and placed in a stereotaxic frame (900SL-KOPF). Ophthalmic eye ointment was applied to the cornea to prevent desiccation during surgery. The area around the incision was trimmed and Vetedine solution (Vétoquinol) was applied. All drugs were injected bilaterally into the dorsal hippocampi using the following coordinates (from Bregma, Paxinos and Franklin, 2008): Medio-lateral X=+/−1.4 mm, Antero-posterior Y=2.0 mm and height Z=−1.33 mm. A 1 μl volume of either AAV or drugs was injected stereotaxically over 4 min (injection speed: 0.25 μl per min) using a 10 μl Hamilton syringe (1701RN). To limit reflux along the injection track, the needle was maintained in situ for 4 min between each 1 μl injection. The skin was closed using silk suture. Behavioral tests were conducted 12 hours or 3 weeks after stereotactic injections. Mice were monitored during recovery.

Pharmacological Modulation of Autophagy

For pharmacological hippocampal induction of autophagy, we performed stereotactic injections of either vehicle (PBS), 1 μg TAT-Beclin 1 (dissolved in PBS) or 1 μg TAT-Scramble (dissolved in PBS). The Tat-Beclin 1 (peptide sequence: YGRKKRRQRRRGGTNVFNATFEIWHDGEFGT, SEQ ID NO:37), consists of 11 amino acids of the TAT protein transduction domain (PTD) at the N terminus, a GG linker to increase flexibility, and 18 amino acids derived from Beclin 1, amino acids 267-284 containing three substitutions: H275E, S279D, Q281E (Provided by Dr. C. Settembre). Control peptide, TAT-Scramble (peptide sequence: YGRKKRRQRRRGGVGNDFFINHETTGFATEW, SEQ ID NO:38), consisted of the TAT protein transduction domain, a GG linker, and a scrambled version of the C-terminal 18 amino acids from Tat-Beclin 1. For pharmacological hippocampal inhibition of autophagy formation, we performed stereotactic injections of either Spautin-1 (5 μg dissolved in DMSO/NaCl (SML0440; Sigma)) or vehicle (DMSO/NaCl (SML0440; Sigma). For pharmacological hippocampal inhibition of the late stage of the autophagic pathway, we performed stereotactic injections of either 100 ng Leupeptin (L2023-50 mg; Sigma) (dissolved in PBS), 50 μg Chloroquine (C6628-25G; Sigma) (dissolved in PBS) or vehicle (PBS). All drugs were infused in a volume of 1 μl (bilaterally) in the dorsal hippocampus (X=+/−1.4 mm, Y=2.0 mm and Z=−1.33 mm). 12 hours after injections mice were subjected to the training phase of the NOR, CFC or OLM behavioral tasks or sacrificed for brain collection.

Adeno-Associated Viruses Expressing shRNA

Adeno-associated viruses (AAV) expressing shRNA were purchased from Vector Biosystems Inc (Malvern Pa.). shRNAs specific to Beclin 1 (Becn 1) (AAV9-GFP-U6-mBECN1-shRNA) (named in the text: AAV-shRNA-Beclin 1), FiP200 (AAV9-GFP-U6-M-RB1CC1-shRNA) (named in the text: AAV-shRNA-Fip200) or scrambled non-targeting negative control (AAV-GFP-U6-scrmb-shRNA) (named in the text: AAV-Scramble) were injected in a volume of 1 μl (bilaterally), 3 weeks prior to behavioral tests or brain tissue collection. The AAV titers were between 2.8 and 4.6×10¹³ GC/ml.

Western Blot Analysis

Mouse hippocampi and cerebellum were dissected, snap frozen and lysed in RIPA lysis buffer (25 mM Tris HCl, pH 7.6, 150 mM NaCl, 1% NP40, 1% Na deoxycholate, 0.1% SDS and cOmplete protease inhibitors (Roche)). The samples were quantified using the Pierce 660 nm Assay, and lysates were mixed with 5.5× sample buffer (11% SDS, 1.4M saccharose, 0.5M Tris, pH 6.8, 10 mg/mL bromophenol blue and DTT 1M), loaded on a 12% SDS polyacrylamide gradient gel and subsequently transferred onto a polyvinylidene difluoride (PVDF) membrane (Biorad). The blots were blocked in Tris-buffered saline with Tween (TBST)-5% BSA and incubated with either mouse anti-β-Actin (1:5000, A-2228, Sigma), mouse anti-Beclin 1 (1:1000, 612113, BD Transduction Laboratories), rabbit anti-LC3 (1:10000, L7543, Sigma), mouse anti-ATGS (1:500, 0262-100/ATGS-7C6, Nanotools), and rabbit anti-VPS34 (1:1000; 4263, Cell Signaling). Horseradish peroxidase-conjugated secondary antibodies (anti-mouse IgG, HRP-linked antibody (7076, Cell Signaling) and anti-rabbit IgG, HRP-linked antibody (7074, Cell Signaling) and revealed using an ECL kit (Clarity Western ECL Substrate, BioRad) for protein detection. Multiple exposures were taken to select images within the dynamic range of the film (GE Healthcare Amersham Hyperfilm ECL). Selected films were scanned and quantified using BioRad Image Lab software (Version 5.2). β-Actin bands were used for normalization.

Semi-Quantitative RT-PCR

Brain tissues were immediately flash-frozen after dissection and total RNA was isolated with Trizol Reagent (Cat. No. 15596) using a homogenizer (OMNI TH). Single-strand cDNA was synthesized from total RNA (2 μg) by using SuperScript II Reverse Transcriptase (ThermoFisher Cat. No. 180640). qRT-PCR was performed using iTAQ SYBR Green (iTaq™ Universal SYBR® Green Supermix, 172-5124, BioRad). Primers were designed and used.

Primary Cultured Hippocampal Neurons

Hippocampi from E16.5 embryos were dissected in L15 cold media. Cells were dissociated chemically in Trypsine-EDTA 0.05% and mechanically by pipetting and then suspended in DMEM (Dulbecco's Modified Eagle Medium) supplemented with 10% fetal bovine serum, and 1% penicillin-streptomycin. All cell culture reagents were purchased from Thermo Fischer Scientific. The dissociated cells were plated onto poly-L-lysine-coated plates or glass coverslips for microscopic examination. 24h after plating, the media was replaced with Neurobasal medium containing B27 supplement, Glutamax and Mycozap. Half of the media was replaced twice a week and neurons were maintained in 5% CO2 and 37° C. until DIV15.

Lentiviral Infections and Transfection of Primary Hippocampal Neurons

Primary hippocampal neurons were infected (MOI5) at Day In Vitro (DIV) 1 with lentiviruses (pLKO-IPTG-3XLacO) expressing an IPTG (Isopropyl β-D-1-thiogalactopyranoside)-inducible shRNA targeting mouse Beclin-1 (Becn 1) (Sigma-Aldrich): 5′-TGC GGG AGT ATA GTG AGT TTA ATT CAA GAG ATT AAA CTC ACT ATA CTC CCG CTT TTT TC-3′ (sense, SEQ ID NO:39); 3′-TCG AGA AAA AAG CGG GAG TAT AGT GAG TTT AAT CTC TTG AAT TAA ACT CAC TAT ACT CCC GCA-5′(anti-sense, SEQ ID NO:40). The same lentiviral plasmid construct together with a plasmid expressing EGFP were also used to co-transfect neurons at DIV11 with Lipofectamine 2000 (Thermo Fischer Scientific) following manufacturer's instructions, to study dendritic spines density. Infected or transfected neurons were treated with 5 mM IPTG (Promega) for 72 hours to induce shRNA-Beclin-1 expression. Neurons were then treated at DIV15 as described below.

Neuronal Stimulation Treatment of Primary Hippocampal Neurons

For chemical long-term potentiation (cLTP), primary hippocampal neurons (DIV15) were treated as described by (Oh et al., 2005). For KCl depolarization, neurons were pretreated with neurobasal medium containing 60 nM KCL for 10 min and for bafilomycin treatment, neurons were treated with neurobasal medium containing 100 mM bafilomycin for 2 hours. After treatment, neurons were rinsed in PBS and proteins extracted in 1× Laemli buffer containing phosphatase and protease inhibitors.

Dendritic Spines Density Analysis In Vitro

Neurons were treated as described in (20) and fixed 1 h after cLTP induction in 4% PFA /4% glucose for 20 min at room temperature. The coverslips were then washed 3 times in PBS and mounted with Fluoromount™ Aqueous Mounting Medium. Lad (Millipore) detection by immunofluorescence was carried out to identify EGFP and shRNA Beclin1 co-transfected neurons. Fluorescence images of Lad and EGFP positive neurons were obtained using a Zeiss Apotome2 (40× objective). Dendritic spine density was analyzed using NeuronStudio software (Rodriguez A. et al 2008). For each neuron, spines from two distinct secondary and tertiary dendrite segments were counted. 16 neurons from 4 biological replicates were analyzed for each group. The analysis was performed blinded by two independent investigators (M.R.B and M.R).

Golgi Cox Staining and Dendritic Spines Quantification

3 month-old treated mice were intracardially perfused with 4% PFA and the isolated brains were subjected to Golgi impregnation was using FD Rapid GolgiStain Kit (FD Neuro Technologies, Baltimore, Md.) following manufacturer's instructions. Bright field z-stacks of 100 μm sections were obtained using a Zeiss Apotome2 (40× objective). Dendritic spine density was quantified manually (ImageJ software, U.S. National Institutes of Health, Bethesda, Md., USA, http://imagej.nih.gov/ij/, 1997-2014). For each mouse group, two secondary dendrite segments from 20-30 of dentate gyrus granule cells were quantified. The analysis was performed blinded by three independent investigators (M.R.B, S.M. and M.R.).

SQSTM1/p62 Puncta Immunostaining and Quantification

Mice were deeply anesthetized with a mixture Ketamine/Xylasine and transcardially perfused with cold PBS, followed by cold 4% PFA. Brains were post-fixed overnight in 4% PFA at 4° C. 30 μm serial coronal floating sections were obtained using a vibratome. Sections were washed with PBS and blocked with 10% fetal bovine serum for 30 min at room temperature. Sections were then incubated with guinea pig anti-p62 (1:200; GP62C, Progen) overnight at 4° C. The sections were washed with PBS before and after being incubated with an Alexa Fluor-conjugated secondary antibodies (donkey anti-guinea pig IgG (Alexa Fluor 488), Life Technologies, 1:200) for 1 hour at room temperature in blocking buffer. All sections were mounted onto gelatin-subbed slides and coverslipped using Mowiol with DAPI. Images were obtained using a Zeiss Apotome.2 fluorescence microscope (20× and 40× objectives). Image analysis was performed using Zen light Zeiss LSM software. The number of cells with SQSTM1/p62 puncta were quantified on digital images with Icy software (http://icy.bioimageanalysis.org).

Electrophysiology

All recordings were carried out blind to experimental conditions. Male C57BL/6J 3 month-old mice perfused with cold artificial cerebrospinal fluid (aCSF) containing (in mM): 128 NaCl, 3 KCl, 1.25 NaH2PO4, 10 D-glucose, 24 NaHCO₃, 2 CaCl2, and 2 MgCl2 (oxygenated with 95% 02 and 5% CO2, pH 7.35, 295-305 mOsm). Acute brain slices containing the CA3 were cut using a microslicer (DTK-1000, Ted Pella) in sucrose-ACSF, which was derived by fully replacing NaCl with 254 mM sucrose, and saturated by 95% 02 and 5% CO2. Slices were maintained in the holding chamber for 1 hr at 37° C. Slices were transferred into the recording chamber fitted with a constant flow rate of ACSF equilibrated with 95% O2/5% CO (2.5 ml/min) at 35° C. Glass microelectrodes (2-4 MΩ) filled with an internal solution containing (mM): 115 potassium gluconate, 20 KCl, 1.5 MgCl2, 10 phosphocreatine, 10 HEPES, 2 magnesium ATP, and 0.5 GTP (pH 7.2, 285 mOsm) were used. Cell excitability was measured with 2 s incremental steps of current injections (50, 100, 150, and 200 pA) at −60 mV holding potential. Series resistance was monitored during all recordings at the beginning and end of each recording, and data were rejected if values changed by more than 20%. All data acquisition and on-line analysis of firing rates were collected using a 700B amplifier, Digidata 1322A digitizer and pClamp 10.2 (Molecular Devices). Spontaneous excitatory postsynaptic currents (sEPSCs) were recorded in voltage clamp at a holding potential of −60 mV with series resistance of <6 MΩ, in the presence of picrotoxin (50 μM).

Novel Object Recognition Paradigm

The behavior sessions were recorded with a video camera. The testing arena consisted on two grey plastic boxes (60×40×32 cm). Mice could not contact or see each other during the exposures. The light intensity was equal in all parts of the arena (approximately 20 lx). Two different objects were used, available in triplicate. The objects were (1) a blue ceramic pot (diameter 6.5 cm, maximal height 7.5 cm) and (2) a clear, plastic funnel (diameter 8.5 cm, maximal height 8.5 cm). The objects elicited equal levels of exploration as determined in pilot experiments and training phase. Mice were transported a short distance from the holding mouse facility to the testing room in their home cages and left undisturbed for at least one hour before the beginning of the test.

The NOR paradigm consists of three phases over 3 days: a habituation phase, a training phase, and a testing phase. Mice were always placed in the center of the arena at the start of each exposure. On day 1: the habituation phase, mice were given 5 min to explore the arena, without any objects and were then taken back to their home cage or for stereotactic surgery. On day 2, the training phase (performed 12 hours after stereotactic surgery), mice were allowed to explore, for 10 min, two identical objects arranged in a symmetric opposite position from the center of the arena and were then transported to their home cage. On day 3, the testing phase, mice were given 15 minutes to explore two objects: a familiar object and a novel one, in the same arena, keeping the same object localization. The object that serves as a novel object (either a blue ceramic pot or a plastic funnel), as well as the left/right localization of the objects were counterbalanced within each group. Mice were placed in the center of the arena at the start of each exposure. Between exposures, mice were held individually in standard cages, the objects and arenas were cleaned with phagosphore, and the bedding replaced. The following behaviors were considered as exploration of the objects: sniffing, licking, or touching the object with the nose or with the front legs or directing the nose to the object at a distance ≤1 cm. Investigation was not scored if the mouse was on top of the object or completely immobile. The preference index for the novel object was calculated as (time spent exploring the new object/the total time spent exploring both objects), and the discrimination index was calculated as (time spent exploring the new object−time spent exploring the familiar object)/(total time spent exploring both objects). Behavior was scored on videos by two observers blind to treatment and the total exploration time of the objects was quantified in the training and testing phases (M.G and S.M).

Object Location Memory test

For the Object location memory task, all procedures were identical to the Novel object recognition task except that during the testing phase, rather than presenting a novel object, mice encountered both familiar objects, with one object located in a different place in the arena. The time and frequency of exploration of the novel/relocated object is measured as an index of memory. Behavior was scored on videos by two observers blind to treatment and the total exploration time of the objects was quantified in the training and testing phases (M.G and S.M).

Contextual Fear Conditioning

Mice were transported a short distance from the holding mouse facility to the testing room in their home cages and left undisturbed for at least one hour before the beginning of the test. The conditioning chambers were obtained from Bioseb (France) with internal dimensions of 25×25×25 cm. Each chamber was located inside a larger, insulated plastic cabinet that provided protection from outside light and noise (67×55×50 cm, Bioseb, France), and mice were tested individually in the conditioning boxes. Floors of the chamber consisted of 27 stainless steel bars wired to a shock generator with scrambler for the delivery of foot shock. Signal generated by the mice movements was recorded and analyzed through a high sensitivity weight transducer system. The analog signal was transmitted to the Freezing software module through the load cell unit for recording purposes and analysis of time active/time immobile (Freezing) was performed. The CFC procedure took place over two consecutive days. On day 1, mice were placed in the conditioning chamber, and received 3 foot-shocks (1 sec, 0.5 mA), which were administrated at 60, 120 and 180 sec after the animals were placed in the chamber. They were returned to their home cages 60 sec after the final shock. Contextual fear memory was assessed 24 hours after conditioning by returning the mice to the conditioning chamber and measuring freezing behavior during a 4 min retention test. Freezing was scored and analyzed automatically using Packwin 2.0 software (Bioseb, France). Freezing behavior was considered to occur if the animals froze for a period of at least two seconds. Behavior was scored by the Freezing software and analyzed by two observers blind to mouse treatment or AAV-infections (M.G and S.M).

Morris Water Maze

Animals were transported a short distance from the holding mouse facility to the testing room in their home cages and left undisturbed for at least one hour prior the first trial. Morris water maze (MWM) with an automatic tracking system was employed for assessing spatial learning and memory. The apparatus was a white circular swimming pool (diameter: 200 cm, walls: 60 cm high), which was located in a room with various distal cues. The pool was filled with water (depth: 50 cm) maintained at 22° C.±1° C., which was made opaque by the addition of a nontoxic white paint. A 12 cm round platform was hidden 1.0 cm below the water surface. The maze was virtually divided into four arbitrary, equally spaced quadrants delineated by the cardinal points north (N), east (E), south (S), and west (W). The pool is located in a brightly lit room. Extra maze geometric and high-contrast cues were mounted on the walls of the swimming pool with the ceiling providing illumination. Data was collected using a video camera fixed to the ceiling and connected to a videotracking system (Anymaze). Each daily trial consisted of four swimming trials, in which each mouse was placed in the pool facing the wall of the tank and allowing the animal to swim to the platform before 120 sec had elapsed. A trial terminated when the animal reached the platform, where it remained for 5 sec. Mice were removed and placed back in their home cages for a 5 min inter-trial interval. To prevent hypothermia, the animals were gently dried with a paper towel between and after the trials. The starting point differed at each trial, and different sequences of release points were used from day to day. Swimming time to the platform was calculated as an evaluation of performance of the mice to locate the target. At day 10, animals were given a probe trial, which consisted of letting the mouse swim in the pool for a fixed duration (120 sec), while the platform was removed but with the same distal cues on the wall. The performance in the probe trial was expressed as the time spent in the target quadrant where the platform was located during the hidden platform training. Animal movements were recorded using Anymaze to calculate parameters of the performance of mice. Behavior was scored by two observers blind to treatment (M.G and S.M).

Memory Stimulation Procedure

Animals were either exposed to CFC or MWM to induce memory stimulation adapted from (21, 25). For the CFC, we used one- or four-days for the training phase. For the one-day training, mice were placed into the conditioning chamber, received three shocks at 60, 120 and 180 sec (1 sec, 0.5 mA), and were removed 60 sec following the last shock and returned to their home cages. For the four-day training, this procedure was repeated four times but at a shock intensity of 0.25 mA. Contextual fear memory was assessed 24 hours following training by returning the mice to the same conditioning chamber and measuring the freezing behavior during a 4 min retention test. Freezing was scored and analyzed automatically using Packwin 2.0 software. Freezing behavior was considered to occur if the animal froze for at least a period of two seconds. For the MWM, the mice were subjected to the normal procedure (described above) but for 5 successive days. On the last day of stimulation, the mice were sacrificed 1 hour after the last exposition to CFC or MWM task.

Plasma Collection and Intravenously Injection

Pooled mouse blood was collected from 60 young (8 weeks) and 6 aged (16 months) mice by intracardial bleed at time of euthanasia. Plasma was prepared from blood collected with EDTA into Capiject T-MQK tubes followed by centrifugation at 1,000 g. All plasma aliquots were stored at −80° C. until use. Before administration, plasma was dialyzed using 3.5-kDa Maxi D-tube dialyzers (71508-3, Novagen) in PBS to remove EDTA. 16 month-old mice were injected with isolated plasma (100 μl per injection), by tail vein injection seven times over 24 days. Mice were then subjected to NOR, one day after the last injection.

Statistical Analysis

For all experiments, the effect of treatment was analyzed using one-way ANOVAs and with repeated-measures where appropriate. Significant ANOVAs were also analyzed using Fisher's PLSD tests where appropriate. All main effects and interactions are noted in the text or figures. All data were analyzed using GraphPad Prism v5 software. Results from data analyses are expressed as means±SEM. Alpha was set to 0.05 for all analyses. * p<0.05, ** p<0.01, *** p<0.001.

Results

To explore the role of autophagy in hippocampal neuronal function and behavior, we subjected 3 month-old mice to memory stimulation by the Morris water maze (MWM) or contextual fear conditioning (CFC). Key ATG genes and genes associated with autophagy (Becn 1, Vps34 and Atg5) were increased at both mRNA and protein levels in the hippocampus (data not shown). Moreover, there was an accumulation of the AP marker LC3-II, which together with the decrease in autophagy cargo SQSTM1/p62, suggested that memory stimulation induces autophagic flux in hippocampal neurons (data not shown). To address more directly the role of autophagy in regulating hippocampal-dependent learning and memory, we next performed hippocampal stereotactic injections of Adeno-Associated Viruses (AAV) expressing shRNA against Beclin 1 (Becn 1), a protein engaged in the initiation of autophagy (1, 2). A decrease in the conversion of LC3 (LC3-I) to the AP-associated lipidated form (LC3-II) 3 weeks after injections verified that autophagy had been hampered in the hippocampus (data not shown). The mice were then subjected to behavioral tasks assessing hippocampal-dependent memory. We found that down-regulating Beclin 1 expression impaired memory capacities as measured by the Novel object recognition (NOR) test and CFC (data not shown), two tests that require the integrity of the hippocampus (16). Next, these mice were subjected to the MWM with a hidden platform for 9 successive days, to assess the ability of mice to use spatial cues to locate a submerged platform. Hippocampal down-regulation of Beclin 1 induces a significant delay in spatial learning capacities (data not shown). This was confirmed by a probe trial MWM task 24h after the last day of acquisition that demonstrated no significant preference for the pool quadrant in AAV-shRNA Beclin 1 (data not shown). Of note, re-exposing the mice to the same context 6 days after the last acquisition day showed that Beclin 1 down-regulated mice did not consolidate memory to the same extent as control mice (data not shown). To rule out a possible autophagy-independent role for Beclin 1, we performed NOR and CFC in mice targeted for Fip200, a component of the ULK1/AGT1 complex involved in early AP formation. Fip200 down-regulation with stereotactic injections of AAV-shRNA and a decrease in autophagy were confirmed by measuring Fip200 expression and LC3-II abundance (data not shown). These mice phenocopy the decreases in memory performance observed after Beclin 1 down-regulation in NOR and CFC assays (data not shown). Taken together, these results suggest that the initiation of autophagy is required for the control of hippocampal-dependent learning and memory.

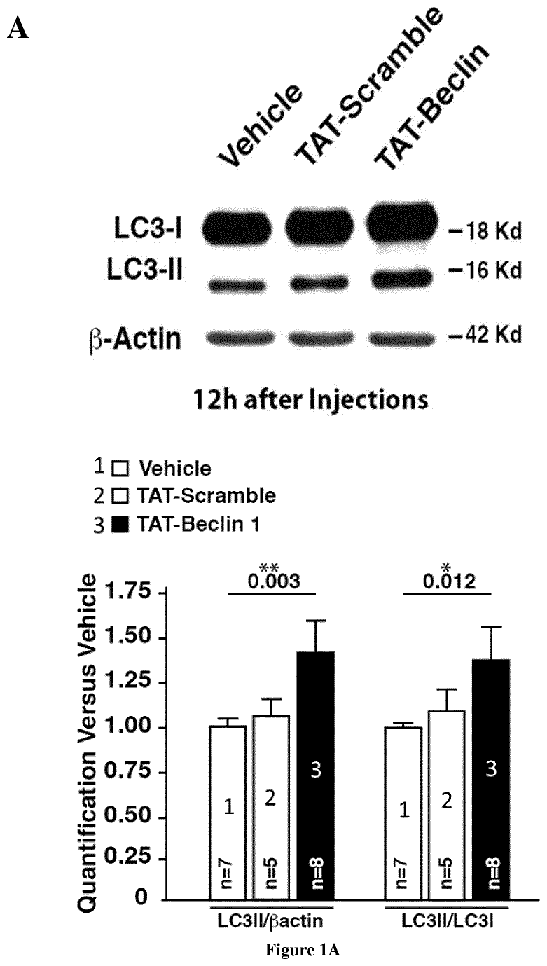

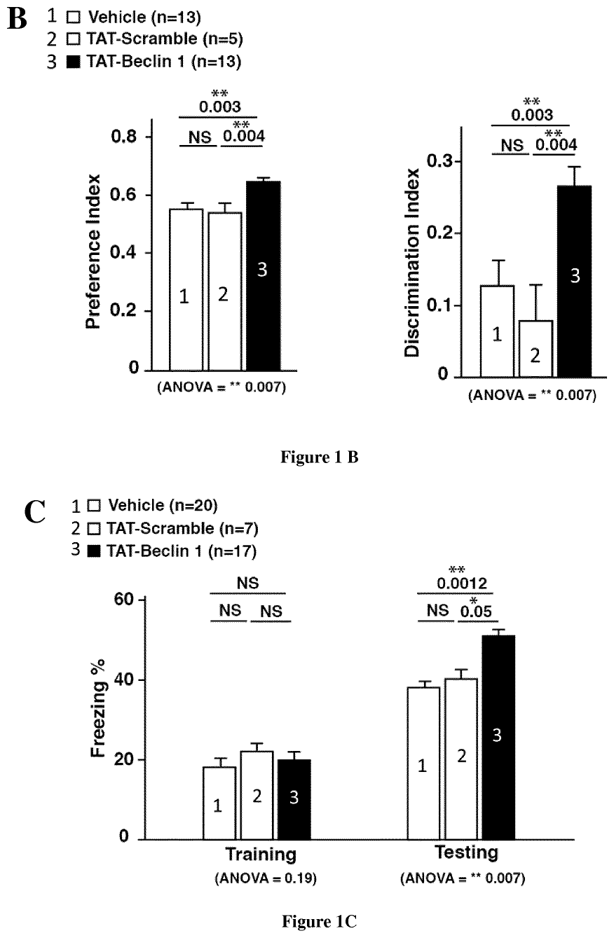

To determine whether autophagy is required for the adaptive response of hippocampal neurons to novel memory stimulation, we acutely modulated autophagy by performing single hippocampal stereotactic injections of inhibitors or inducers of autophagy in 3 month-old WT mice (data not shown). Induction or inhibition of autophagy 12h after acute pharmacological injections was confirmed by measuring LC3-II abundance and SQSTM1/p62 levels in the hippocampus (data not shown). We then subjected them 12 hours later to the training (acquisition) phase of NOR and CFC (data not shown). Acute injections of the Spautin-1 (specific autophagy inhibitor-1) (data not shown), interfering with AP formation, or blocking the late stage of the autophagic pathway by injecting chloroquine or leupeptin (data not shown), all led to a significant decrease in memory performance during the testing phase of both NOR and CFC (data not shown). Conversely, an acute induction of autophagy following hippocampal injections of the TAT-Beclin 1 peptide (17), an inducer of AP formation, during the training phase (FIGS. 1A, B and C), enhanced learning/memory capacities in both tests. Hence, these data indicate that hippocampal autophagy is necessary for the control of novel memory acquisition.

Novel memory acquisition relies on the adaptive response of hippocampal neurons to stimuli, characterized by lasting changes in neuronal morphology, synaptic activity and neurotransmission (5, 10, 18). Long-term potentiation (LTP) is one of the key cellular mechanisms underlying learning and memory (18, 19). Increased synaptic strength following LTP induction results in the rapid formation of new dendritic spines, small actin-rich protrusions extending from dendrites that house excitatory synapses, along with phosphorylation of post-synaptic AMPA glutamate receptors and CAMKII (calcium/calmodulin-dependent protein kinase II) (5, 18). We observed that chemical long-term potentiation (cLTP), which induces long-lasting synaptic plasticity, or KCL depolarization, increases autophagy in mature primary hippocampal neurons, as determined by measuring LC3-II level (data not shown). Therefore, we investigated whether down-regulation of autophagy may influence neuronal plasticity of hippocampal neurons in response to cLTP (20) or memory stimulation induced by MWM (21), which promotes formation of novel dendritic spines in granular neurons of the hippocampal dentate gyrus. In vitro, we performed acute knockdown of Beclin 1 in primary hippocampal neurons by infection with lentiviruses expressing an IPTG inducible-shRNA against Beclin 1. Treatment with IPTG induced a robust decrease in Beclin 1 protein levels (data not shown). Neuronal plasticity induction by cLTP significantly increased novel dendritic spine formation, and phosphorylation of GluA1 receptors (pGluA1) in Ser831 and CAMKII (pCaMKII) in the control group, whereas no significant induction of either novel dendritic spines or synaptic molecular strength in response to cLTP treatment was observed after down-regulation of Beclin 1 (data not shown). This observation suggests that autophagy is required to mediate the adaptability of hippocampal neurons to respond to a long-lasting stimulation. To confirm this observation in vivo, 3 month-old mice, locally injected with either AAV-shRNA Beclin 1 or AAV-shRNA Scramble, were subjected to MWM for 5 consecutive days (22). After Golgi staining, we observed a significant induction of dendritic spine formation in hippocampal granular neurons of control mice (AAV-Scramble-injected), but not in those of the AAV-shRNA Beclin 1-injected mice (data not shown). Accordingly, electrophysiological analyses showed a significant decrease in neuronal excitability (data not shown) and sESPC frequency (data not shown) in hippocampal neurons of the CA3 after local down-regulation of Beclin 1. These data indicate that autophagy in hippocampal neurons is required for an adaptive neuronal response to novel memory stimulations.

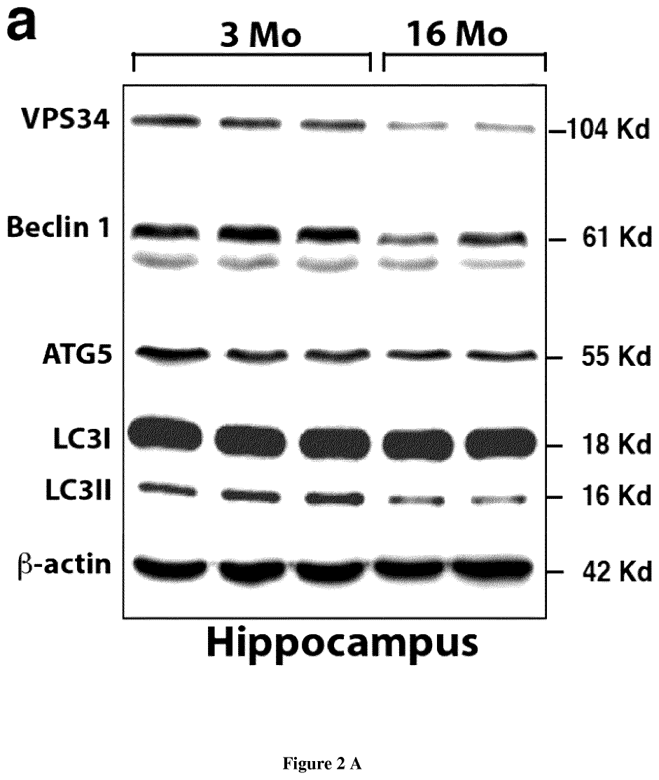

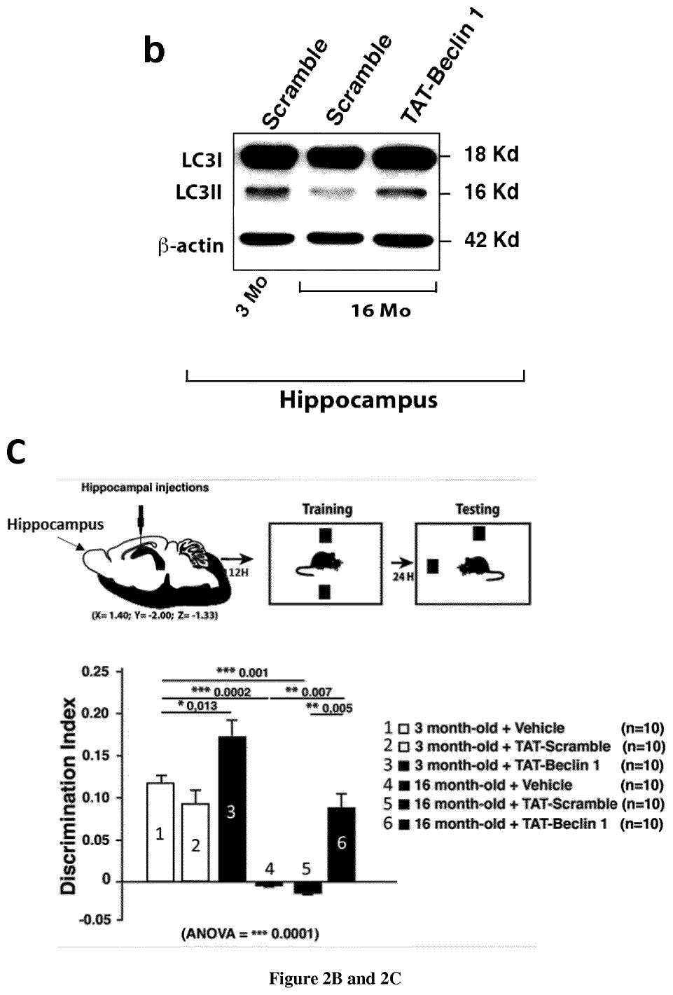

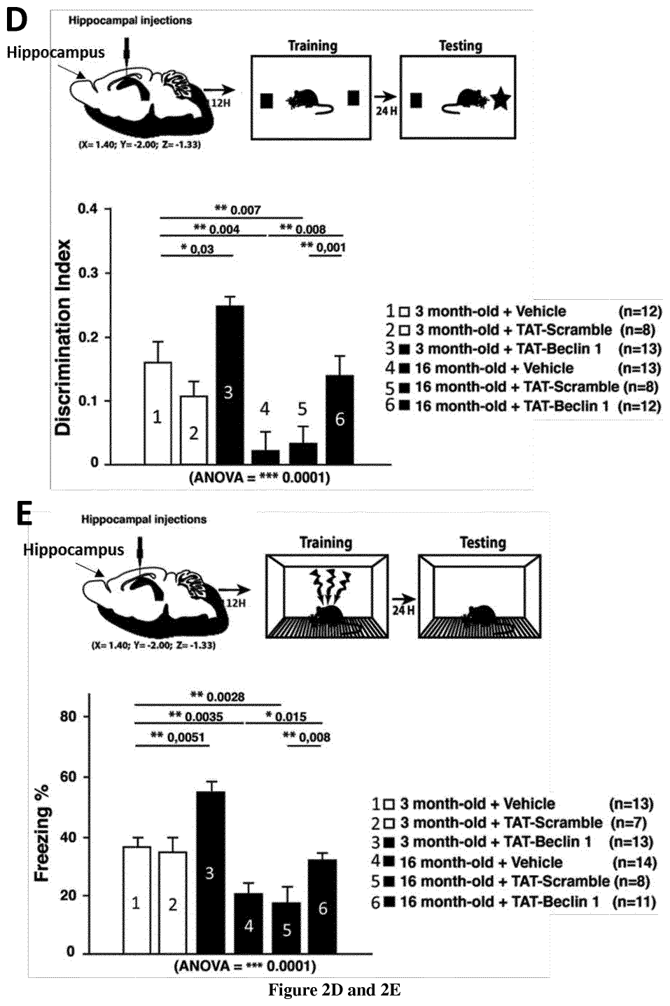

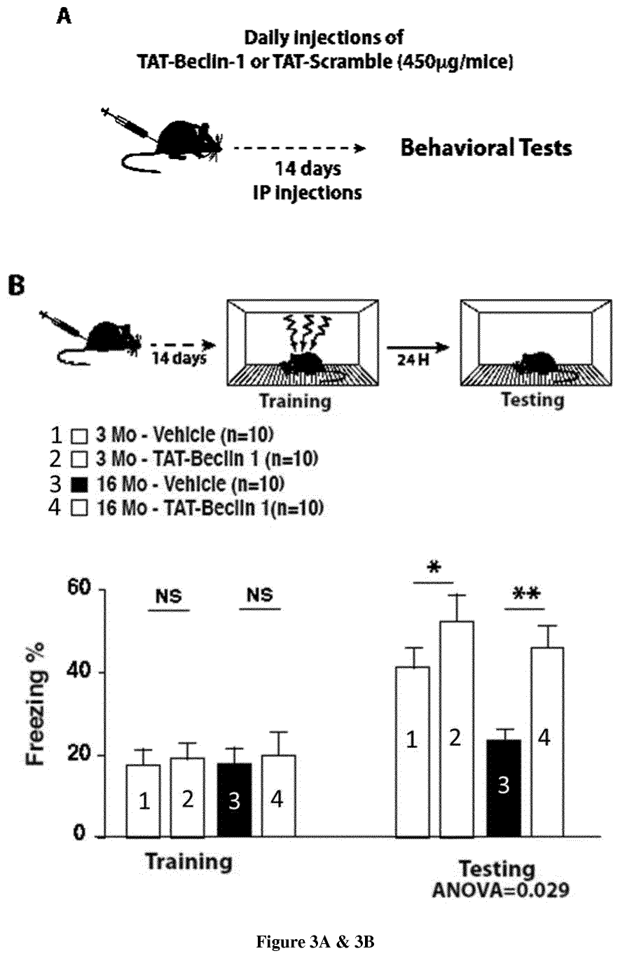

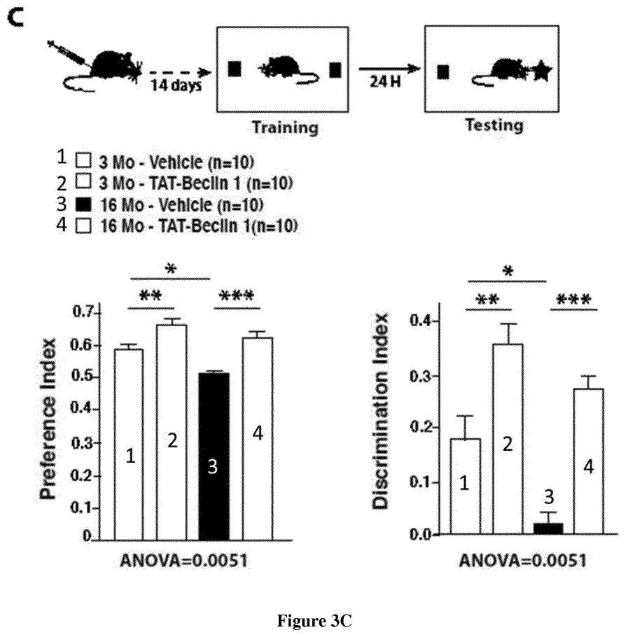

ATG protein levels and autophagic flux are reduced during normal brain aging (3). In agreement with these data, we observed a decrease in Beclin 1, VPS34 and ATG5 at both protein and mRNA levels, and a decrease in LC3-II accumulation together with an increase in SQSTM1/p62 in the hippocampi of 16 month-old WT mice (FIG. 2A). We next tested whether the decrease in hippocampal autophagy during aging may contribute to age-related cognitive decline by subjecting 3 or 16 month-old WT mice to NOR, CFC and Object location memory (OLM) tests after local stereotactic injections of either vehicle, TAT-Scramble or TAT-Beclin 1. We observed that increasing autophagy levels in 16 month-old animals was sufficient to rescue age-related memory deficits and to restore memory to levels seen in 3 month-old mice (FIGS. 2A, 2B, 2C, 2D, 2E, 3A, 3B and 3C). As various environmental factors, such as young circulating factors, can protect against age-related hippocampal-dependent memory and adult neurogenesis decline (23, 24), we hypothesized that hippocampal autophagy could mediate, at least in part, the recently described beneficial effects of young plasma in aged mice on hippocampal-dependent memory. We found that 16 month-old mice receiving injections of plasma collected from 8 week-old mice had increased LC3-II levels, indicating increased autophagy (data not shown) and corrected age-related memory deficits (data not shown). This beneficial effect was not observed when plasma from 2 month-old mice was injected in 16 month-old animals in which Beclin 1 was down-regulated (data not shown).

Our study describes for the first time an important role for autophagy in the brain, for the regulation of hippocampal-dependent memory. It demonstrates that neuronal autophagy has an unprecedented, functionally critical role in the brain rather than only protecting it from energy shortage or degradation of mis-folded proteins. Indeed, hippocampal autophagy is required to mediate the activity-dependent changes of neurons in response to memory stimulation and environmental stimuli. Moreover, our findings demonstrate that restoring autophagy activity (via the TAT-beclin for example) level in old hippocampus could reverse age-related memory deficits. In that respect, our results may have important therapeutic implications to prevent the decline and/or extend resilience of mental health during aging.

### REFERENCES

Throughout this application, various references describe the state of the art to which this invention pertains. The disclosures of these references are hereby incorporated by reference into the present disclosure.
1. N. Mizushima, M. Komatsu, Autophagy: renovation of cells and tissues. Cell 147, 728-741 (2011); published online EpubNov 11 (10.1016/j.cell.2011.10.026).
2. E. Morel, M. Mehrpour, A. C. Nascimbeni, P. Codogno, Autophagy: A Druggable Process. Annual review of pharmacology and toxicology 57, 375-398 (2017); published online EpubJan 06 (10.1146/annurev-pharmtox-010716-104936).
3. D. C. Rubinsztein, G. Marino, G. Kroemer, Autophagy and aging. Cell 146, 682-695 (2011); published online EpubSep 2 (10.1016/j.cell.2011.07.030).
4. R. A. Nixon, The role of autophagy in neurodegenerative disease. Nature medicine 19, 983-997 (2013); published online EpubAug (10.1038/nm.3232).
5. T. Bartsch, P. Wulff, The hippocampus in aging and disease: From plasticity to vulnerability. Neuroscience 309, 1-16 (2015); published online EpubNov 19 (10.1016/j.neuroscience.2015.07.084).
6. D. T. Gray, C. A. Barnes, Distinguishing adaptive plasticity from vulnerability in the aging hippocampus. Neuroscience 309, 17-28 (2015); published online EpubNov 19 (10.1016/j.neuroscience.2015.08.001).
7. B. Leuner, E. Gould, Structural plasticity and hippocampal function. Annual review of psychology 61, 111-140, C111-113 (2010)10.1146/annurev.psych.093008.100359).
8. C. H. Bailey, E. R. Kandel, Structural changes accompanying memory storage. Annual review of physiology 55, 397-426 (1993)10.1146/annurev.ph.55.030193.002145).
9. S. N. Burke, C. A. Barnes, Senescent synapses and hippocampal circuit dynamics. Trends in neurosciences 33, 153-161 (2010); published online EpubMar (10.1016/j.tins.2009.12.003).
10. N. A. Bishop, T. Lu, B. A. Yankner, Neural mechanisms of ageing and cognitive decline. Nature 464, 529-535 (2010); published online EpubMar 25 (10.1038/nature08983).
11. C. N. Harada, M. C. Natelson Love, K. L. Triebel, Normal cognitive aging.

Clinics in geriatric medicine 29, 737-752 (2013); published online EpubNov (10.1016/j.cger.2013.07.002).
12. P. Boya, F. Reggiori, P. Codogno, Emerging regulation and functions of autophagy. Nature cell biology 15, 713-720 (2013); published online EpubJul (10.1038/ncb2788).
13. C. A. Lamb, T. Yoshimori, S. A. Tooze, The autophagosome: origins unknown, biogenesis complex. Nature reviews. Molecular cell biology 14, 759-774 (2013); published online EpubDec (10.1038/nrm3696).
14. A. M. Cuervo, Autophagy and aging: keeping that old broom working. Trends in genetics: TIG 24, 604-612 (2008); published online EpubDec (10.1016/j.tig.2008.10.002).
15. T. T. Ho, M. R. Warr, E. R. Adelman, 0. M. Lansinger, J. Flach, E. V. Verovskaya, M. E. Figueroa, E. Passegue, Autophagy maintains the metabolism and function of young and old stem cells. Nature 543, 205-210 (2017); published online EpubMar 09 (10.1038/nature21388).
16. L. Fioriti, C. Myers, Y. Y. Huang, X. Li, J. S. Stephan, P. Trifilieff, L. Colnaghi, S. Kosmidis, B. Drisaldi, E. Pavlopoulos, E. R. Kandel, The Persistence of Hippocampal-Based Memory Requires Protein Synthesis Mediated by the Prion-like Protein CPEB3. Neuron 86, 1433-1448 (2015); published online EpubJun 17 (10.1016/j.neuron.2015.05.021).
17. S. Shoji-Kawata, R. Sumpter, M. Leveno, G. R. Campbell, D. J. Lenschow, A. Yamamoto, D. King, O. Lichtarge, N. V. Grishin, S. A. Spector, D. V. Kaloyanova, B. Levine, Identification of a candidate therapeutic autophagy-inducing peptide. Nature 494, 201-206 (2013); published online EpubFeb 14 (10.1038/nature11866).
18. V. A. Derkach, M. C. Oh, E. S. Guire, T. R. Soderling, Regulatory mechanisms of AMPA receptors in synaptic plasticity. Nature reviews. Neuroscience 8, 101-113 (2007); published online EpubFeb (10.1038/nrn2055).
19. T. V. Bliss, G. L. Collingridge, A synaptic model of memory: long-term potentiation in the hippocampus. Nature 361, 31-39 (1993); published online EpubJan 07 (10.1038/361031a0).
20. E. Menna, S. Zambetti, R. Morini, A. Donzelli, L. Pattini, C. Frassoni, M. Francolini, G. Scita, M. Sala, M. Fahnestock, M. Matteoli, Eps8 controls dendritic spine density and synaptic plasticity through its actin-capping activity. The EMBO journal 32, 1730-1744 (2013); published online EpubJun 12 (10.1038/emboj.2013.107).
21. X. Jiang, G. S. Chai, Z. H. Wang, Y., Q. Wang, J. Z. Wang, G. P. Liu, Spatial training preserves associative memory capacity with augmentation of dendrite ramification and spine generation in Tg2576 mice. Scientific reports 5, 9488 (2015); published online EpubMar 30 (10.1038/srep09488).
22. R. R. Mahmmoud, S. Sase, Y. D. Aher, H. Hoger, G. Lubec, Spatial and Working Memory Is Linked to Spine Density and Mushroom Spines. PloS one 10, e0139739 (2015)10.1371/journal.pone.0139739).
23. S. A. Villeda, J. Luo, K. I. Mosher, B. Zou, M. Britschgi, G. Bieri, T. M. Stan, N. Fainberg, Z. Ding, A. Eggel, K. M. Lucin, E. Czirr, J. S. Park, S. Couillard-Despres, L. Aigner, G. Li, X. S. Xie, T. A. Rando, T. Wyss-Coray, The ageing systemic milieu negatively regulates neurogenesis and cognitive function. Nature 477, 90-94 (2011); published online EpubSep 1 (10.1038/nature10357).
24. S. A. Villeda, K. E. Plambeck, J. Middeldorp, J. M. Castellano, K. I. Mosher, J. Bieri, B. Zou, D. A. Simmons, X. S. Xie, F. M. Longo, T. Wyss-Coray, Young blood reverses age-related impairments in cognitive function and synaptic plasticity in mice. Nature medicine 20, 659-663 (2014); published online EpubJun (10.1038/nm.3569).
25. J. Sanders, K. Cowansage, K. Baumgartel, M. Mayford, Elimination of dendritic spines with long-term memory is specific to active circuits. The Journal of neuroscience: the official journal of the Society for Neuroscience 32, 12570-12578 (2012); published online EpubSep 05 (10.1523/JNEUROSCI.1131-12.2012).

Ya-ping YANG, Li-fang HU, Hui-fen ZHENG, Cheng-jie MAO, Wei-dong HU, Kang-ping XIONG, Fen WANG, Chun-feng LIU. Application and interpretation of current autophagy inhibitors and activators. Acta Pharmacologica Sinica (2013) 34: 625-635.

1. (canceled)

2. (canceled)

3. (canceled)

4. (canceled)

5. The method of claim 11, wherein the activator is selected from the group consisting of Earle's balanced salt solution (EBSS), Brefeldin A, Thapsigargin, Tunicamycin, Rapamycin, CCI-779, RAD001, AP23576, Small molecule enhancers rapamycin (SMER), Trehalose, L-690,330, Carbamazepine, Valproic acid sodium salt, N-Acetyl-D-sphingosine (C2-ceramide), Penitrem A, Calpastatin, Xestospongin B, Akebia saponin, Amiodarone hydrochloride, ATG13, GF 109203X synthetic, GF 109203X hydrochloride, N-Hexanoyl-D-sphingosine, MRT68921 dihydrochloride, Niclosamide, Qc1, Rottlerin, STF-62247, Tamoxifen, Temsirolimus, ULK Active, Z36 and Hydroxycitrate.

6. The method of claim 11, wherein the activator is Beclin 1.

7. The method of claim 11, wherein the activator is a peptide comprising an amino acid sequence of formula (I) (SEQ ID NO: 2):
Xaa1-N-A-T-F-Xaa2-Xaa3-Xaa4-Xaa5,
wherein:
Xaa1, Xaa2, Xaa3, Xaa4 and Xaa5 are each an amino acid independently selected from the group consisting of Alanine (A), Arginine (R), Asparagine (N), Aspartic acid (D), Cysteine (C), Glutamic acid (E), Glutamine (Q), Glycine (G), Histidine (H), Isoleucine (I), Leucine (L), Lysine (K), Methionine (M), Phenylalanine (F), Proline (P), Serine (S), Threonine (T), Tryptophan (W), Tyrosine (Y), Valine (V), allyl glycine (AllylGly), norleucine, norvaline, biphenylalanine (Bip), citrulline (Cit), 4-guanidinophenylalanine (Phe(Gu)), homoarginine (hArg), homolysine (hLys), 2-naphtylalanine (2-Nal), ornithine (Orn) Of and pentafluorophenylalanine.

8. The method of claim 7 wherein the activator is the peptide TAT-Beclin 1 of SEQ ID NO:37.

9. (canceled)

10. (canceled)

11. A method of i) restoring and/or improving one or more cognitive functions, ii) treating a cognitive disease, iii) preventing or reversing the deleterious effects of aging on cognitive function, and/or iv) restoring and/or improving age-related memory decline or age-related memory loss in a subject in need thereof, comprising administering to the subject a therapeutically effective amount of an activator of autophagy.
